(19) United States
(12) Patent Application Publication
(10) Pub. No.: US 2013/0045160 A1
(43) Pub. Date: Feb. 21, 2013

(54) Magnetic Nano-Composite for Contrast Agent, Intelligent Contrast Agent, Drug Delivery Agent for Simultaneous Diagnosis and Treatment, and Separation Agent for Target Substance

(71) Applicants: Seung-Joo HAM, Seoul (KR); Jin-Suck SUH, Seoul (KR); Yong-Min HUH, Seoul (KR); Ho-Geun YOON, Gyeonggi-do (KR); Jae-Moon YANG, Gyeonggi-do (KR)

(72) Inventors: Seung-Joo HAM, Seoul (KR); Jin-Suck SUH, Seoul (KR); Yong-Min HUH, Seoul (KR); Ho-Geun YOON, Gyeonggi-do (KR); Jae-Moon YANG, Gyeonggi-do (KR)

(73) Assignee: Industry-Academic Coorporation Foundation, Yonsei University, Seoul (KR)

(21) Appl. No.: 13/590,958

(22) Filed: Aug. 21, 2012

Related U.S. Application Data
(63) Continuation of application No. 12/280,474, filed on Dec. 3, 2008, now abandoned.

(30) Foreign Application Priority Data
Feb. 24, 2006 (KR) 10-2006-0018469

(51) Int. Cl. A61K 9/51; A61K 39/00; A61K 38/17; A61K 51/12; A61K 38/19; A61K 38/20; A61K 38/21; A61K 38/49; A61P 9/00; A61P 1/00; A61P 25/00; A61K 39/395; A61K 38/18; B82Y 5/00

(52) U.S. Cl. 424/1.11; 424/490; 424/178.1; 424/193.1; 514/1.1; 424/85.1; 514/7.6; 424/85.2; 424/85.4; 424/94.64; 977/773; 977/810

(57) ABSTRACT

The present invention relates to water soluble magnetic nanocomposite using an amphiphilic compound. Specifically, the present invention relates to water soluble magnetic nanocomposite which may be not only used as a contrast agent for magnetic resonance imaging (MRI), an intelligent contrast agent for diagnosing cancer characterized by binding a tissue-specific binder ingredient, a drug delivery system for simultaneous diagnosis and treatment by polymerizing or enveloping drugs and binding a tissue-specific binder ingredient, but also used for separating a target substance using magnetism, and a process for preparing the same.

### CROSS-REFERENCE TO RELATED APPLICATIONS

The present application is a continuation application of U.S. patent application Ser. No. 12/280,474, filed Dec. 3, 2008, which is a national stage application under 35 U.S.C. §371 of PCT Application No. PCT/KR2007/000961 having an international filing date of Feb. 23, 2007, which claims priority to Korean Application Serial No. 10-2006-0018469, filed Feb. 24, 2006, all of which are expressly incorporated herein by reference in their entireties.

### TECHNICAL FIELD

The present invention relates to a water soluble magnetic nanocomposite using an amphiphilic compound. Specifically, the present invention relates to a water soluble magnetic nanocomposite which may be not only used as a contrast agent for magnetic resonance imaging (MRI), an intelligent contrast agent for diagnosing cancer characterized by binding a tissue-specific binder ingredient, a drug delivery system for simultaneous diagnosis and treatment by polymerizing or enclosing a drug and binding a tissue-specific binder ingredient, but also used for separating a target substance using magnetism, and a process for preparing the same

### BACKGROUND ART

Nanotechnology is the technique of manipulating and controlling materials on an atomic or molecular scale, is suitable to invent new materials or new devices, and thus has a variety of applications such as electronics, materials, communication, mechanics, medicine, agriculture, energy, and environment Nanotechnology is variously developed and is classified as the following three fields:

First, it relates to the technique of synthesizing new materials with an ultramicroscopic size as nano-materials.

Second, it relates to the technique of manufacturing devices, such as nano devices, displaying a certain function by combining or arranging materials with a nano scale.

Third, it relates to the technique of applying the nanotechnology, called nanobiotechnology, to biotechnology.

Especially, among the field of nanobiotechnology, the magnetic nanoparticle is used in a broad range of applications, such as separation of biological components, a diagnostic probe for magnetic resonance imaging, biosensors including giant magnetoresistive sensors, micro fluidic sensors, drug/gene delivery, and a magnetic hyperthermia.

In particular, the magnetic nanoparticle may be used in a diagnostic probe (contrast agent) of molecular magnetic resonance imaging. The magnetic nanoparticle is allowed to reduce a spin-spin relaxation time of a hydrogen atom in water molecules surrounding nanoparticles to show the effect of amplifying signals of magnetic resonance imaging, and thus have been broadly used in diagnosis of resonance imaging.

In addition, the magnetic nanoparticle may serve as a probe material of Giant magnetic resistance (GMR) sensor. When the magnetic nanoparticle senses a biological molecule patterned on the surface of GMR biosensor and binds to it, it changes current signals of the GMR sensor. Using such change, a biological molecule can be selectively detected (U.S. Pat. No. 6,452,763 B1; U.S. Pat. No. 6,940,277 B2; U.S. Pat. No. 6,944,939 B2; U.S. 2003/0133232 A1).

Furthermore, the magnetic nanoparticle may be applied to separate a biological molecule. For example, when a cell expressing specific biomarker is mixed with other cells, only the desired cell may be separated along direction of the magnetic field by selectively binding the nanoparticle to a specific biomarker and then applying an external magnetic field (Whitehead et al. U.S. Pat. No. 4,554,088,U.S. Pat. No. 5,665,582, U.S. Pat. No. 5,508,164, U.S. 2005/0215687 A1). Furthermore, it may be applied to separate various biological molecules, including a protein, an antigen, a peptide, DNA, RNA, and a virus as well as a cell. Also, the magnetic nanoparticle may be applied to micro fluidic censors to separate and detect biological molecules. It is possible to detect and separate a biological molecule in a micro unit system by forming very small channels on a chip and a flowing magnetic nanoparticle therein.

Meanwhile, the magnetic nanoparticle may also be used in a biotherapy through delivering a drug or a gene. The selective effect of treatment may be obtained by moving the nanoparticle loaded with a drug or a gene through a chemical bond or adsorption to the desired position by an external magnetic field and allowed to release the drug and the gene on the region of interest (U.S. Pat. No. 6,855,749).

As another example of application for biotherapy using the magnetic nanoparticle, it includes hyperthermia using magnetic spin energy (U.S. Pat. No. 6,530,944 B2, U.S. Pat. No. 5,411,730). Along an external alternating current with radio frequency on the magnetic nanoparticle, heat is released through a spin flipping procedure. If the temperature around nanoparticle is more than 40° C., a cell is killed due to high heat and thus a disease cell may be selectively killed.

To apply the magnetic nanoparticle for the uses described above, it should have an excellent magnetic property, be stably carried and dispersed in vivo, that is, in a water soluble environment, and be capable to easily combine with a bioactive material. A variety of techniques have been developed until now to meet such conditions.

U.S. Pat. No. 6,274,121 relates to a paramagnetic nanoparticle comprising a metal such as iron oxide and discloses a nanoparticle to whose surface is bound to an inorganic substance including binding sites for coupling to a tissue-specific binding substance, a diagnostic or pharmacologically active substance.

U.S. Pat. No. 6,638,494 relates to a paramagnetic nanoparticle comprising a metal such as iron oxide and discloses the method of preventing aggregation and sedimentation of a nanoparticle in a gravitational field or in a magnetic field by binding a particular carboxylic acid to its surface. As said carboxylic acid, aliphatic dicarboxylic acid such as maleic acid, tartaric acid, or glucaric acid, or aliphatic poly dicarboxylic acid such as citric acid, citric acid, cyclohexane, or tricarboxylic acid was used.

U.S. Patent Application Publication No. 2004/58457 relates to a functionalized nanoparticle coated with a monolayer. A bifunctional peptide is attached to said monolayer, to which various biopolymers including DNA and RNA may be bound.

GB Patent Application No. 223,127 relates to a method for making a magnetic nanoparticle, including the step of forming a magnetic nanoparticle within a protein template, wherein described a method for encapsulating a nanoparticle into apoferritin.

U.S. Patent Application Publication No. 2003/190,471 relates to a method for forming a nanoparticle of manganese zinc ferrite within dual micelles, wherein was described the nanoparticle showing an improved property through procedures of heat treating the formed magnetic nanoparticle.

In U.S. Patent Application Publication No. 2005/130,167, the synthesis of a water soluble magnetic nanoparticle covered with 16-mercaptohexadecanoic acid was described, together with detection of a virus and mRNA in an experimental rat with intracellular magnetic labeling, using a TAT peptide, a transfection agent, on the synthesized magnetic nanoparticle.

KR Patent Application No. 10-1998-0705262 discloses a particle comprising a superparamagnetic iron oxide core provided with a starch coating and optionally a polyalkyleneoxide coating and MRI contrast media containing the same.

However, water soluble nanoparticles prepared by the methods above have the following disadvantages:

In U.S. Pat. Nos. 6,274,121, 6,638,494; U.S. Patent Application Publication Nos. 2003/190,471, 2004/58457 and 2005/130,167, GB Patent Application No. 223,127, and KR Patent Application No. 10-1998-0705262, since the disclosed nanoparticle is mainly synthesized in a water solution, the size of nanoparticle is not easily controlled and the resulting nanoparticle show a non-uniform size distribution. In addition, since they are synthesized at a low temperature, the resulting nanoparticle has low crystalline property, and can be formed in a non-stoichiometric compound. Therefore, the nanoparticles prepared by the methods above have problems that show low stability of colloid in a water solution and thus aggregation on applying in vivo, and high non-selective binding, and the like.

### DISCLOSURE

### Technical Problem

The present invention intends to solve the problems above. The object of the present invention is to provide a magnetic nanocomposite having so high stability in a water solution with low toxicity that may widely apply for diagnosis and treatment of organism, which is characterized in that a magnetic nanoparticle is covered with an amphiphilic compound having one or more hydrophobic domains and one or more hydrophillic domains.

Another object of the present invention is to provide an intelligent magnetic nanocomposite which is characterized in that a magnetic nanoparticle is covered with an amphiphilic compound having one or more hydrophobic domains and one or more hydrophillic domains, and one or more binding parts for a hydrophillic active ingredient present in said hydrophillic domain are bound to a tissue-specific binding substance.

Still another object of the present invention is to provide a magnetic nanocomposite for simultaneous diagnosis and treatment which is characterized in that a magnetic nanoparticle is covered with an amphiphilic compound having one or more hydrophobic domains and one or more hydrophillic domains, one or more binding parts for a hydrophillic active ingredient present in said hydrophillic domain are bound to a tissue-specific binding substance, and a pharmaceutically active ingredient is bound to or enclosed in said hydrophobic domain.

Still another object of the present invention is to provide a method for separating a target substance which comprises binding a magnetic nanocomposite to a target substance and applying a magnetic field on the combination of the magnetic nanocomposite and the target substance, wherein said nanocomposite is characterized in that a magnetic nanoparticle is covered with an amphiphilic compound having one or more hydrophobic domains and one or more hydrophillic domains, and one or more binding parts for a hydrophillic active ingredient present in said hydrophillic domain are bound to a tissue-specific binding substance.

Still another object of the present invention is to provide a method for preparing the magnetic nanoparticle according to the present invention above.

The other object of the present invention is to provide a contrast agent, a composition for diagnosis and a pharmaceutical composition, comprising the magnetic nanocomposite according to the present invention above and a pharmaceutically acceptable carrier, and a method for using the same.

### Technical Solution

The magnetic nanocomposite according to the present invention is described in more detail below.

The magnetic nanocomposite according to the present invention has one feature that an amphiphilic compound is added to a surface of nanoparticle to bind the hydrophobic domains of amphiphilic compounds to the surface of nanoparticle, and to distribute the hydrophillic domains of an amphiphilic compound over the outermost part of nanoparticle. The hydrophobic domains of an amphiphilic compound are bound to the surface of nanoparticle by a hydrogen bond, Van der Waals force, and a physical bond such as a polar attraction. Therefore, said hydrophobic domain does not only play a role in dispersing a nanoparticle in matrix of the hydrophobic domain or binding to the surface of nanoparticle, but also, if necessary, may physically enclose a drug in the matrix of hydrophobic domain or chemically bind a drug to one end of the hydrophobic domain. Meanwhile, the hydrophillic domain of an amphiphilic compound may be distributed in the outermost part of nanocomposite to stabilize a water insoluble nanoparticle in a water soluble medium and maximize bioavailability.

In addition, the magnetic nanocomposite according to the present invention has another feature that a metal, a magnetic material, or a magnetic alloy as a nanoparticle may be bound to an organic surface stabilizer. The bond of metal, magnetic material, or magnetic alloy to the organic surface stabilizer is achieved by coordinating the organic surface stabilizer to a precursor of metal, magnetic material, or magnetic alloy to form a complex. Said organic surface stabilizer may act in stabilizing the hydrophobic domain of an amphiphilic compound.

Furthermore, the magnetic nanocomposite according to the present invention has another feature that said hydrophobic domain may have one or more binding parts for a hydrophobic active ingredient (R1) within some part of its structure, and said hydrophilic domain may have one or more binding parts for a hydrophilic active ingredient (R2) within some part of its structure. When various active ingredients are bound to the binding parts for a hydrophilic active ingredient and the binding parts for a hydrophobic active ingredient, the magnetic nanocomposite according to the present invention may be used in various uses such as an intelligent contrast agent for cancer diagnosis, a drug delivery system that cancer diagnosis and treatment can be simultaneously performed, and an agent for separating a protein. Its schematic diagram is depicted in FIG. 1.

As shown in FIG. 2, the magnetic nanocomposite according to the present invention as above includes a magnetic nanocomposite comprising a core that one or more magnetic nanoparticles are distributed in the hydrophobic domain and a shell containing the hydrophilic domain (“emulsion type magnetic nanocomposite,” below) and a magnetic nanocomposite comprising a core that one magnetic nanoparticle is bound to the hydrophobic domains and a shell containing the hydrophilic domain (“suspension type magnetic nanocomposite,” below), depending on their preparation methods.

It is preferred that all the magnetic nanoparticles of the emulsion type magnetic nanocomposite and suspension type magnetic nanocomposite is coordinated to a metal, a magnetic material, or a magnetic alloy, and that magnetic nanoparticles are physically bound to the hydrophobic domains of an amphiphilic compound.

In addition, the desired diameter of the emulsion type nanocomposite is 1 nm to 500 nm, and more preferably 25 nm to 100 nm. The desired diameter of the suspension type nanocomposite is 1 nm to 50 nm, and more preferably 5 nm to 30 nm.

It is also preferred the magnetic nanocomposite according to the present invention that a magnetic nanoparticle is covered with an amphiphilic compound having one or more hydrophobic domains and one or more hydrophilic domains, and one or more binding parts for a hydrophilic active ingredient are bound to a tissue-specific binding substance.

It is also preferred the magnetic nanocomposite according to the present invention that a magnetic nanoparticle is covered with an amphiphilic compound having one or more hydrophobic domains and one or more hydrophilic domains, one or more binding parts for a hydrophilic active ingredient are bound to a tissue-specific binding substance, and a pharmaceutically active ingredient is bound or enclosed in the hydrophobic domains.

“Magnetic nanoparticle” in the magnetic nanocomposite according to the present invention may be used without limitation, as long as it has magnetism and have a diameter of 1 nm to 1000 nm, and preferably 2 nm to 100 nm, but it is preferably a metal material, a magnetic material, or a magnetic alloy.

Said metal is not specifically limited, but preferably selected from the group consisting of Pt, Pd, Ag, Cu and Au.

Said magnetic material is also not specifically limited, but preferably selected from the group consisting of Co, Mn, Fe, Ni, Gd, Mo, MM′₂O₄, and MₓO_{y} (each M or M′ independently represents Co, Fe, Ni, Mn, Zn, Gd, or Cr, 0<x=3, 0<y=5).

In addition, said magnetic alloy is also not specifically limited, but preferably selected from the group consisting of CoCu, CoPt, FePt, CoSm, NiFe and NiFeCo.

It is also preferred that a metal, a magnetic material, or a magnetic alloy is bound to an organic surface stabilizer. The organic surface stabilizer is referred to an organic functional molecule being capable to stabilize the state or size of nanoparticle, and includes a surfactant as a representative example.

The surfactant that may be used includes, but not limited to, cationic surfactant, including alkyl trimethylammonium halide; neutral surfactant, including saturated or unsaturated fatty acid such as oleic acid, lauric acid, or dodecylic acid, trialkylphosphine or trialkylphosphine oxide such as trioctylphosphine oxide (TOPO), trioctylphosphine (TOP), or tributylphosphine, alkyl amine such as dodecylamine, oleicamine, trioctylamine, or octylamine, or alkyl thiol; and anionic surfactant, including sodium alkyl phosphate.

Especially, considering stabilization and uniform size distribution of nanoparticles, it is preferred to use a saturated or unsaturated fatty acid and/or alkylamine.

The amphiphilic compound according to the present invention is not specifically limited, if it has one or more hydrophobic domains (P1) and one or more hydrophilic domains (P2). In the amphiphilic compound, the hydrophobic domains (P1) and a hydrophilic domains (P2) may be linked and bounded as multi domains. That is, the amphiphilic compound may have a variety of forms such as P1-P2, P1-P2-P1, P2-P1-P2, P1-(P2-P1)n-P2, P1-(P2-P1)n-P1, P2-(P1-P2)n-P1, or P2-(P1-P2)n-P2. Of course, the repeated hydrophobic domains or hydrophilic domains may be present within its structure.

The hydrophobic domains of an amphiphilic compound according to the present invention may consist of a compound or a polymer. For example, a biocompatible saturated or unsaturated fatty acid, or a hydrophobic polymer may be used.

Said saturated fatty acid is not specifically limited, but may use one or more selected from the group consisting of butyric acid, caproic acid, caprylic acid, capric acid, lauric acid (dodecyl acid), miristic acid, palmitic acid, stearic acid, eicosanoic acid, and docosanoic acid. Said unsaturated fatty acid is also not specifically limited, but may use one or more selected from the group consisting of oleic acid, linoleic acid, linolenic acid, arakydonic acid, eicosapentanoic acid, docosahexanoic acid, and erucic acid.

Saturated or unsaturated fatty acids that may be used in the amphiphilic compound according to the present invention are set forth in Tables 1 and 2 below:

TABLE 1

| Chemical Name | Formula | Length of carbon chain |
|---|---|---|
| Butyric (butanoic acid) | CH₃(CH₂)₂COOH | C4 |
| Caproic (hexanoic acid) | CH₃(CH₂)₄COOH | C6 |
| Caprylic (octanoic acid) | CH₃(CH₂)₆COOH | C8 |
| Capric (decanoic acid) | CH₃(CH₂)₈COOH | C10 |
| Lauric (dodecanoic acid) | CH₃(CH₂)₁₀COOH | C12 |
| Myristic (tetradecanoic | CH₃(CH₂)₁₂COOH | C14 |
| acid) | | |
| Palmitic (hexadecanoic | CH₃(CH₂)₁₄COOH | C16 |
| acid) | | |
| Stearic (octadecanoic acid) | CH₃(CH₂)₁₆COOH | C18 |
| Arachidic (eicosanoic acid) | CH₃(CH₂)₁₈COOH | C20 |
| Behenic (docosanoic acid) | CH₃(CH₂)₂₀COOH | C22 |

TABLE 2

| | | Length of carbon |
|---|---|---|
| | | chain:Number of |
| Chemical Name | Formula | double bond |
| Oleic acid | CH₃(CH₂)₇CH═CH(CH₂)₇COOH | C18:1 |
| Linoleic acid | CH₃(CH₂)₄CH═CHCH₂CH═CH(CH₂)₇COOH | C18:2 |
| Alpha-linolenic | CH₃(CH₂)CH(═CHCH₂CH═)₂CH(CH₂)₇COOH | C18:3 |
| acid | | |
| Arachidonic acid | CH₃(CH₂)₄CH(═CHCH₂CH)₃═CH(CH₂)₃COOH | C20:4 |
| Eicosapentaenoic | CH₃CH₂CH(═CHCH₂CH)₄═CH(CH₂)₃COOH | C20:5 |
| acid | | |
| Docosahexaenoic | CH₃(CH₂)CH(═CHCH₂CH)₅═CHCH₂CH₂COOH | C22:6 |
| acid | | |
| Erucic acid | CH₃(CH₂)₇CH═CH(CH₂)₁₁COOH | C22:1 |

Meanwhile, the hydrophobic polymer that may be used in the amphiphilic compound according to the present invention is not specifically limited, but preferably one or more selected from the group consisting of polyphospazene, polylactide, polylactide-co-glycolide, polycaprolactone, poly anhydride, polymalic acid or derivatives thereof, polyalkylcyanoacrylate, polyhydroxybutylate, polycarbonate, polyorthoester, a hydrophobic polyamino acid and a hydrophobic vinyl based polymer. In addition, said hydrophobic polymer has preferably a weight average molecular weight of 100 to 100,000. If the weight average molecular weight is less than 100, toxicity of the polymer is occurred. If the weight average molecular weight is in excess of 100,000, it is difficult to be applied.

The hydrophilic domains of an amphiphilic compound according to the present invention may consist of a compound or a polymer. For example, a biocompatible polymer may be used.

Said biocompatible polymer is not specifically limited, but preferably one or more selected from the group consisting of polyalkyleneglycol (PAG), polyetherimide (PEI), polyvinylpyrrolidone (PVP), a hydrophilic polyamino acid and a hydrophilic vinyl based polymer, and more preferably polyethyleneglycol. In addition, said hydrophilic polymer has preferably a weight average molecular weight of 100 to 100,000. If the weight average molecular weight is less than 100, toxicity of the polymer is occurred. If the weight average molecular weight is in excess of 100,000, it is difficult to be applied.

Especially, said polyalkyleneglycol is preferably polyethyleneglycol (PEG) or monomethoxypolyethyleneglycol (mPEG), and more preferably polyethyleneglycol substituted with carboxyl or amine.

In addition, said hydrophobic domain (P1) has one or more binding parts for a hydrophobic active ingredient (R1) within some part of its structure, preferably in the end of structure. Said hydrophilic domain (P2) has one or more binding parts for a hydrophilic active ingredient (R2) within some part of its structure, preferably in the end of structure.

When a material that may specifically be bound to, for example, a tumor marker is bound to the binding parts for a hydrophilic ingredient (R2), the magnetic nanocomposite according to the present invention may be used in an intelligent conatrast agent for cancer diagnosis.

When a drug is polymerized or enclosed in the binding parts for a hydrophobic active ingredient (R1) or the hydrophobic domains (P1), and the material that may specifically be bound to a tumor marker is simultaneously bound to the binding parts for a hydrophilic ingredient (R2), the magnetic nanocomposite according to the present invention may be used in a drug delivery system for simultaneous diagnosis and treatment of cancer.

Meanwhile, when an antibody or a protein specific to a surface antigen of functional cell, stem cell or cancer cell is bound to the binding parts for a hydrophilic active ingredient (R2), the magnetic nanocomposite according to the present invention may be used for separating a cell and a protein using magnetism.

In addition, said hydrophilic domain (P2) of the magnetic nanocomposite according to the present invention is characterized in that the domain has the binding parts for a hydrophilic active ingredient (R2) within its structure, preferably in the end of structure, and the binding parts for a hydrophilic active ingredient (R2) are bound to a tissue-specific binding substance.

Said hydrophilic active ingredient may be selected from the group consisting of a bioactive ingredient, a polymer, and an inorganic support. In the specification of the present invention, “a bioactive ingredient” has the same meaning as “a tissue-specific binding substance” or “a pharmaceutically active ingredient,” which may be used interchangeably each other.

The binding part for a hydrophilic active ingredient (R2) may be optionally changed depending on a hydrophilic active ingredient, that is, a tissue-specific binding substance, to be bound. Preferably, the binding part includes, but not limited to, one or more functional groups selected from the group consisting of —COOH, —CHO, —NH₂, —SH, —CONH₂, —PO₃H, —PO₄H, —SO₃H, —SO₄H, —OH, —NR₄+X—, -sulfonate, -nitrate, -phosphonate, -succinimidyl, -maleimide, and -alkyl.

The tissue-specific binding substance includes, but not limited to, an antigen, an antibody, RNA, DNA, hapten, avidin, streptavidin, neutravidin, protein A, protein G, lectin, selectin, a radioisotope labeled component, or a tumor marker.

The nanocomposite of the present invention may be used for diagnosing and/or treating various diseases related to tumor, for example, gastric cancer, lung cancer, breast cancer, ovarian cancer, liver cancer, bronchial cancer, nasopharyngeal cancer, laryngeal cancer, pancreatic cancer, bladder cancer, colon cancer and cervical cancer.

Such tumor cell expresses and/or secretes particular materials less or not at all produced by a normal cell, which generally called “tumor marker.” The nanocomposite prepared by binding a material that may be specifically bound to such tumor marker to the binding parts for an active ingredient of the water soluble nanoparticle may be advantageously used in diagnosing tumor. Not only various tumor markers but also materials that may be specifically bound to such tumor marker are known in this field.

In addition, the tumor marker may be classified as a ligand, an antigen, a receptor, and encoding nucleic acids thereof, depending on the mode of action.

TABLE 3

| Class | Example of tumor marker | Example of active ingredient |
|---|---|---|
| Ligand | C2 of cynaptotagmin I | Phosphatidylserine |
| | annexin V | |
| | Intergrin | Integrin receptor |
| | VEGF | VEGFR |
| | angiopoietin 1, 2 | Tie2 receptor |
| | Somatostatin | somatostatin receptor |
| | vasointestinal peptide | vasointestinal peptide receptor |
| Antigen | carcinoembryonic antigen | Herceptin (Genentech, USA) |
| | HER2/neu antigen | Herceptin (Genentech, USA) |
| | prostate-specific antigen | Rituxan (Genentech, USA) |
| Receptor | Follic acid receptor | Follic acid |

When the tumor marker is a ligand, the material that may be specifically bound to said ligand can be introduced as an active ingredient of the nanocomposite according to the present invention, and suitably, a receptor or an antibody that may be specifically bound to the ligand. Examples of a ligand to be used herein and a receptor that may be specifically bound to the ligands include, but not limited to, C2 of synaptotagmin and phosphatidylserine, annexin V and phosphatidylserine, integrin and receptor thereof, VEGF (Vascular Endothelial Growth Factor) and receptor thereof, angiopoietin and a Tie2 receptor, somatostatin and receptor thereof, a vasointestinal peptide and receptor thereof, and the like.

When the tumor marker is an antigen, the material that may be specifically bound to the antigen can be introduced as an active ingredient of nanocomposite according to the present invention, and suitably, an antibody that may be specifically bound to the antigen. Examples of an antigen to be used herein and an antibody that may be specifically bound to the antigen include a carcinoembryonic antigen (colon cancer labeled antigen) and Herceptin (Genentech, USA), a HER2/neu antigen (breast cancer labeled antigen) and Herceptin, a prostate-specific membrane antigen (prostate cancer labeled antigen) and Rituxan (IDCE/Genentech, USA), and the like.

Representative examples of “a receptor” as the tumor marker include a folic acid receptor expressed in ovarian cancer. The material that may be specifically bound to the receptor (folic acid in case of folic acid receptor) can be introduced as an active ingredient of nanocomposite according to the present invention, and suitably, a ligand or an antibody that may be specifically bound to the receptor.

As described above, an antibody as an active ingredient is most preferably herein, because the antibody has a property being selectively and stably bound to the particular subject only and —NH₂ of lysine, —SH of cysteine, and —COOH of asparginic acid and glutamic acid present in Fc domain of the antibody may be usefully utilized to be bound to a functional group of binding parts for an active ingredient in a water soluble nanocomposite.

Such antibody is commercially available or may be prepared according to the known methods in this field. Generally, a mammal (for example, mouse, rat, goat, rabbit, horse or sheep) is immunized more than one time with an appropriate amount of an antigen. After a certain time period, when the titer reaches to the appropriate level, the antibody is recovered from serum of the mammal. If desired, the recovered antibody may be purified using the known process and stored in the frozen buffer solution until use. Detail of such method is well known in this field.

Meanwhile, said “nucleic acid” includes a ligand, an antigen, a receptor or RNA and DNA encoding some of these, as described above. As known in this field, a nucleic acid is characterized by forming base pairs between complementary sequences. Thus, the nucleic acid having particular base sequences may be detected, using the nucleic acid having complementary base sequences to said base sequences. The nucleic acid having complementary base sequences to the nucleic acid encoding an enzyme, a ligand, an antigen, a receptor above may be used as an active ingredient of nanocomposite according to the present invention.

In addition, the nucleic acid has a functional group such as —NH₂, —SH, —COOH on the 5′- and 3′-ends, and thus may be usefully used to be bound to the functional group of binding parts for an active ingredient.

Such nucleic acid can be synthesized by the standard method known in this field, for example, using an automatic DNA synthesizer (for example, those available from Biosearch, Applied Biosystems, and the like). For example, phosphorothioate oligonucleotide may be synthesized by the method described in Stein et al. Nucl. Acids Res. 1988, vol. 16, p. 3209. Methylphosphonate oligonucleotide may be synthesized using the controlled glass polymer support (Sarin et al. Proc. Natl. Acad. Sci. U.S.A. 1988, vol. 85, p. 7448).

Meanwhile, it is preferred that the hydrophobic domain (P1) of the magnetic nanocomposite according to the present invention has one or more binding parts for a hydrophobic active ingredient (R1) within some part of its structure, preferably, in the end of structure. The hydrophobic active ingredient may be bound or enclosed in said binding parts for a hydrophobic active ingredient (R1) or said hydrophobic domains (P1).

The hydrophobic active ingredient is preferably selected from the group consisting of a bioactive ingredient, a polymer, and an inorganic support. For example, when a drug as a hydrophobic active ingredient is bound or enclosed, and a tissue-specific binding substance is simultaneously bound, to the binding parts for a hydrophilic active ingredient (R2), the magnetic nanocomposite may be used in a drug delivery system for simultaneous diagnosis and treatment of cancer.

The binding parts for a hydrophobic active ingredient (R1) in said hydrophobic domain (P1) may be optionally changed depending on the kind of hydrophobic active ingredient to be bound. Preferably, representative examples include, but not limited to, one or more functional groups selected from the group consisting of —COOH, —CHO, —NH₂, —SH, —CONH₂, —PO₃H, —PO₄H, —SO₃H, —SO₄H, —OH, -succinimidyl, -maleimide, and -alkyl.

The hydrophobic active ingredient is not specifically limited, if it is a pharmaceutically active ingredient, but preferably one or more selected from the group consisting of an anticancer agent, an antibiotic, a hormone, a hormone antagonist, interleukin, interferon, a growth factor, a tumor necrosis factor, endotoxin, lymphotoxin, eurokinase, streptokinase, a tissue plasminogen activator, a protease inhibitor, alkylphosphocholine, a radioisotope labeled component, a surfactant, a cardiovascular system drug, a gastrointestinal system drug and a nervous system drug.

Meanwhile, the hydrophobic active ingredient present in the hydrophobic domain, particularly, an anticancer agent may be enclosed by a physical inclusion, a chemical inclusion, or a combination thereof. The inclusion of a drug is achieved through a physical bond of an anticancer agent with a hydrophobic active ingredient of an amphiphilic polymer, for preparing magnetic nanocomposite by an emulsion method and a suspension method. In case of an anticancer agent which can be bound to binding parts for a hydrophobic active ingredient of an amphiphilic polymer constituting a magnetic nanocomposite, it may be bound to binding parts for a hydrophobic active ingredient of the amphiphilic polymer by an appropriate cross-linking agent and thus the inclusion of a drug in the magnetic nanocomposite can be achieved.

The anticancer agent which may be used in the method of treatment according the present invention includes, but not limited to, Epirubicin, Docetaxel, Gemcitabine, Paclitaxel, Cisplatin, Carboplatin, Taxol, Procarbazine, Cyclophosphamide, Dactinomycin, Daunorubicin, Etoposide, Tamoxifen, Doxorubicin, Mitomycin, Bleomycin, Plicomycin, Transplatinum, Vinblastin and Methotrexate.

In the magnetic nanocomposite according to the present invention, it is preferred that an amphiphilic compound consists of a hydrophobic domain—a hydrophilic domain, or a hydrophilic domain—a hydrophobic domain—a hydrophilic domain. When each binding part for an active ingredient is included in hydrophilic domains and hydrophobic domains, the amphiphilic compound may consist of binding parts for a hydrophobic active ingredient—a hydrophobic domain—a hydrophilic domain—a binding part for a hydrophilic active ingredient, or binding parts for a hydrophilic active ingredient—a hydrophilic domain—a hydrophobic domain(—a binding part for a hydrophobic active ingredient)—a hydrophilic domain—a binding part for a hydrophilic active ingredient. Especially, it is preferred to have a functional group such as —NH₂— in the hydrophilic domains and hydrophobic domains, such as binding parts for a hydrophobic active ingredient—a hydrophobic domain —NH₂— a hydrophilic domain—a binding part for a hydrophilic active ingredient. —NH₂— group present in the hydrophilic domains and hydrophobic domains may have more stable structure, if an amphiphilic compound is added to a surface of magnetic nanoparticle.

Most preferably, examples of an amphiphilic compound in the magnetic nanocomposite according to the present invention include carboxylpolyethyleneglycol-polylactide-co-glycolide copolymer or poly(ethylene oxide)-poly(propylene oxide)-poly(ethylene oxide) copolymer substituted with carboxy groups on both ends.

The present invention also relates to a method for preparing a magnetic nanocomposite which comprises the steps of:
A) synthesizing nanoparticles in a solvent; and
B) adding an amphiphilic compound having a hydrophobic domain and a hydrophilic domain to the surfaces of nanoparticles to bind the amphiphilic compound and nanoparticles.

The method for preparing the magnetic nanocomposite according to the present invention further comprises optionally
C) binding the binding part present in said hydrophilic domain and the material that may be specifically bound to a tumor marker; and
D) binding or enclosing a pharmaceutically active ingredient in the hydrophobic domain.

The method for preparing the magnetic nanocomposite according to the present invention is described in more detail below.

The step A) of synthesizing nanoparticles in a solvent is one that precursors of nanoparticles are reacted with a surface stabilizer, and preferably includes the steps of
a) reacting an organic surface stabilizer with the precursors of nanoparticles in presence of a solvent; and
b) thermolyzing the resulting reactant.

In the step a), the precursors of nanoparticle are poured into the solvent including an organic surface stabilizer, which is subsequently coordinated to the surfaces of nanoparticles.

As a nanoparticle in the step a), a metal, a magnetic material, or a magnetic alloy is preferably used. The organic surface stabilizer may be selected from the group consisting of alkyl trimethylammonium halide, saturated or unsaturated fatty acid, trialkylphosphine oxide, alkyl amine, alkyl thiol, sodium alkyl sulfate, and sodium alkyl phosphate. The specific example of a metal, a magnetic material, a magnetic alloy and an organic surface stabilizer is described above.

As a precursor of nanoparticle in the step a), a metal compound that the metal is bound to —CO, —NO, —O₅H₅, alkoxides or other known ligands may be used. Specifically, various organic metal compounds may be used, including a metal carbonyl based compound such as iron pentacarbonyl (Fe(CO)₅), ferrocene, or manganese carbonyl (Mn₂(CO)₁₀; or a metal acetylacetonate based compound such as iron acetylacetonate (Fe(acac)₃). A metal ion containing metal salt that the metal is bound to a known anion such as Cl—, or NO₃ may be also used as a precursor of nanoparticle. Specifically, trichloroiron (FeCl₃), dichloroiron (FeCl₂), or iron nitrate (Fe(NO₃)₃) may be used. Furthermore, a mixture of at least two metal precursors mentioned above may be used in synthesizing an alloy nanoparticle and a combined nanoparticle.

Preferably, the solvent that may be used in the step a) has high boiling point attaching to the thermolysis temperature of complex that the organic surface stabilizer is coordinated to the surface of nanoparticle. For example, the solvent selected from the group consisting of an ether compound, a heterocyclic compound, an aromatic compound, a sulfoxide compound, an amide compound, an alcohol, a hydrocarbon and water may be used.

Specifically, the usable solvent is an ether compound such as octyl ether, butyl ether, hexyl ether, or decyl ether; a heterocyclic compound such as pyridine, or tetrahydrofuran (THF); an aromatic compound such as toluene, xylene, mesitylen, or benzene; a sulfoxide compound such as dimethylsulfoxide (DMSO); an amide compound such as dimethylformamide (DMF); an alcohol such as octyl alcohol, or decanol; a hydrocarbon such as pentane, hexane, heptane, octane, decane, dodecane, tetradecane, or hexadecane; or water.

The reaction conditions in the step a) are not specifically limited, and may be suitably regulated depending on the kinds of metal precursor and surface stabilizer. The reaction may be occurred at room temperature or below. Usually, it is preferred to heat and keep the step in the range of about 30 to 200° C.

In the step b), the complex that the organic surface stabilizer is coordinated to the surface of nanoparticle is thermolyzed to grow nanoparticle. According to the reaction conditions, a metal nanoparticle with uniform size and shape may be formed. The thermolysis temperature may be also suitably regulated depending on the kinds of metal precursor and surface stabilizer. Preferably, the reaction is suitably subjected in a range of about 50 to 500° C. The nanoparticle prepared in the step b) may be separated and purified by the known means.

In the method for preparing a magnetic nanocomposite according to the present invention, the step B) comprises adding the amphiphilic compound having hydrophobic domains and a hydrophilic domains to the surface of nanoparticle to bond the amphiphilic compound and the nanoparticle.

The method of adding the amphiphilic compound to the surface of magnetic nanoparticle is classified into an emulsion type and a suspension type as described above, whose schematic diagram is set forth in FIG. 2.

More specifically, the adding step B) preferably comprises the steps of:
a) dissolving a nanoparticle in an organic solvent to prepare an oil phase;
b) dissolving an amphiphilic compound in an aqueous solvent to prepare an aqueous phase;
c) mixing the oil phase with the aqueous phase to form an emulsion; and
d) separating the oil phase from the emulsion. The emulsion type magnetic nanocomposite according to the present invention may be prepared.

In addition, the adding step B) preferably comprises the steps of:
e) dispersing a nanoparticle in a solution dissolving an amphiphilic compound to prepare a suspension; and
f) separating the solvent from the suspension. The suspension type magnetic nanocomposite according to the present invention may be prepared.

In the adding step B), the hydrophobic domain is preferably a saturated or unsaturated fatty acid or a hydrophobic polymer, and the hydrophilic domain is preferably a biodegradable polymer. Specific example is described above.

In the adding step B), the amphiphilic compound may be prepared by the known methods in this field. For example, it may be prepared by polymerizing diamine polyethylene glycol (NH₂—PEG-NH₂) constituting the hydrophilic group and polylactide-co-glycolide, a biodegradable polymer, constituting the hydrophobic group.

In addition, the biding part for a hydrophilic active ingredient may be substituted with a succinimidyl group, using N,N′-disuccinimidyl carbonate in the binding part for a hydrophilic active ingredient substituted with an amine group of the amphiphilic polymer. The biding part for a hydrophilic active ingredient of the amphiphilic polymer may be also substituted with a carboxyl group by polymerizing carboxyl/amine polyethylene glycol (NH₂-PEG-COOH) constituting the hydrophilic group and polylactide-co-glycolide, a biodegaradable polymer, constituting the hydrophobic group. The biodegradable amphiphilic polymer may be prepared by ring-opening polymerization using lactide as a monomer. Polymerization of lactide is initiated by an amine group of carboxyl/amine polyethylene glycol. Stannous octoate may be used as a catalyst. The polymerization may be carried out in a temperature of 100 to 180° C. under nitrogen atmosphere. By regulating a molecular weight and an amount of carboxyl/amine polyethylene glycol, an initial macroinitiator, the molecular weight of copolymer may be regulated.

Preferably, the step C), for binding the material to which the binding part for a hydrophilic active ingredient present in the hydrophilic domain and a tumor marker may be specifically bound, comprises the steps of:
g) providing some of the hydrophilic domain with the binding part for a hydrophilic active ingredient, using a cross linking agent; and
h) binding the binding part for a hydrophilic active ingredient and the material that may specifically bind to a tumor marker.

In the step g), the cross linking agent to be used is not specifically limited, but preferably includes one or more selected from the group consisting of 1,4-Diisothiocyanatobenzene, 1,4-Phenylene diisocyanate, 1,6-Diisocyanatohexane, 4-(4-Maleimidophenyl)butyric acid N-hydroxysuccinimide ester, Phosgene solution, 4-(Maleinimido)phenyl isocyanate, 1,6-Hexanediamine, p-Nitrophenyl chloroformate, N-Hydroxysuccinimide, 1,3-Dicyclohexylcarbodiimide, 1,1′-Carbonyldiimidazole, 3-Maleimidobenzoic acid N-hydroxysuccinimide ester, Ethylenediamine, Bis(4-nitrophenyl)carbonate, Succinyl chloride, N-(3-Dimethylaminopropyl)-N′-ethylcarbodiimide Hydrochloride, N,N′-Disuccinimidyl carbonate, N-Succinimidyl 3-(2-pyridyldithio)propionate, and succinic anhydride. The cross linking agent is reacted with some of the hydrophilic domain to provide the binding part for a hydrophilic active ingredient such as —COOH, —CHO, —NH₂, —SH, —CONH₂, —PO₃H, —PO₄H, —SO₃H, —SO₄H, —OH, —NR₄+X—, -sulfonate, -nitrate, -phosphonate, -succinimidyl, -maleimide, or -alkyl.

In the step h), the functional group of binding part for a hydrophilic active ingredient may be changed depending on the kind of active ingredient, that is, a tissue-specific binding component, and its chemical formula.

In the method for preparing a nanocomposite according to the present invention, the step D) for binding or enclosing the pharmaceutically active ingredient in the hydrophobic domain can be classified into a step of physically enclosing the pharmaceutically active ingredient in the hydrophobic domain and a step of chemically binding the pharmaceutically active ingredient to the hydrophobic domain.

Preferably, the chemically binding step comprises the steps of:
i) providing some of the hydrophobic domain with the binding part for a hydrophobic active ingredient, using a cross linking agent; and
j) binding the binding part for a hydrophobic active ingredient and the pharmaceutically active ingredient.

As the cross linking agent that may be used in the step i), the cross linking agent in the step g) above may be employed, without limitation. The cross linking agent is reacted with some of the hydrophobic domain to provide the binding part for a hydrophobic active ingredient such as —COOH, —CHO, —NH₂, —SH, —CONH₂, PO₃H, —PO₄H, —SO₃H, —SO₄H, —OH, -succinimidyl, -maleimide, or -alkyl above.

The step of physical inclusion may be carried out by dissolving the pharmaceutically active ingredient along with nanoparticles and enclosing the ingredient in them in the step B) for binding the amphiphilic compound and nanoparticles. More specifically, when the pharmaceutically active ingredient is enclosed in the emulsion type nanocomposite, the pharmaceutically active ingredient may be physically enclosed in the hydrophobic domain by dissolving the pharmaceutically active ingredient in an organic solvent along with nanoparticles, mixing with the aqueous phase to form an emulsion, and separating the oil phase, in the step a) that the nanoparticles above are dissolved in an organic solvent to prepare the oil phase. When the pharmaceutically active ingredient is enclosed in the suspension type nanocomposite, the pharmaceutically active ingredient may be physically enclosed in the hydrophobic domain by dispersing the pharmaceutically active ingredient along with nanoparticles to prepare suspension and separating the solvent, in the step e) for preparing suspension by dispersing nanoparticles in the solution dissolving the amphiphilic compound.

The bond of binding part for a hydrophilic or hydrophobic active ingredient and a hydrophilic or hydrophobic active ingredient in the steps h) and i) may be changed depending on the kind of each active ingredient, and its chemical formula. Specific example is set forth in Table 4 below.

TABLE 4

| I | II | III |
|---|---|---|
| R—NH₂ | R′—COOH | R—NHCO—R′ |
| R—SH | R′—SH | R—SS—R |
| R—OH | R′-(epoxy) | R—OCH₂C(OH)CH₂—R′ |
| RH—NH₂ | R′-(epoxy) | R—NHCH₂C(OH)CH₂—R′ |
| R—SH | R′-(epoxy) | R—SCH₂C(OH)CH₂—R′ |
| R—NH₂ | R′—COH | R—N═CH—R′ |
| R—NH₂ | R′—NCO | R—NHCONH—R′ |
| R—NH₂ | R′—NCS | R—NHCSNH—R′ |
| R—SH | R′—COCH₂ | R′—COCH₂S—R |
| R—SH | R′—O(C═O)X | R—OCH₂(C═O)O—R′ |
| R-(aziridine) | R′—SH | R—CH₂CH(NH₂)CH₂S—R′ |
| R—CH═CH₂ | R′—SH | R—CH₂CHS—R′ |
| R—OH | R′—NCO | R′—NHCOO—R |
| R—SH | R′—COCH₂X | R—SCH₂CO—R′ |
| R—NH₂ | R′—CON₃ | R—NHCO—R′ |
| R—COOH | R′—COOH | R—(C═O)O(C═O)—R′ + H₂O |
| R—SH | R′—X | R—S—R′ |
| R—NH₂ | R′CH₂C(NH²⁺)OCH₃ | R—NHC(NH²⁺)CH₂—R′ |
| R—OP(O²⁻)OH | R′—NH₂ | R—OP(O²⁻)NH—R′ |
| R—CONHNH₂ | R′—COH | R—CONHN═CH—R′ |
| R—NH₂ | R′—SH | R—NHCO(CH₂)₂SS—R′ |

I: functional group of binding part for an active ingredient
II: active ingredient
III: binding example according to a reaction of I and II

The resulting water soluble nanocomposite in the steps A), B), C) and D) above can be separated using the known methods in this field. Generally, the water soluble nanocomposite is produced in a precipitate. Thus, it is preferred to separate it using centrifugation or filtration.

The present invention further relates to a contrast agent comprising a magnetic nanocomposite using an amphiphilic compound and a pharmaceutically acceptable carrier; a composition for diagnosing disease comprising a conjugate of a tissue-specific binding ingredient and the magnetic nanocomposite, and a pharmaceutically acceptable carrier; a pharmaceutical composition for simultaneous diagnosis and treatment comprising a conjugate of a tissue-specific binding ingredient and a pharmaceutically active ingredient and the magnetic nanocomposite, and a pharmaceutically acceptable carrier.

The carrier used in the composition according to the present invention includes carriers and vehicles usually used in the pharmaceutical field. Specifically, it includes, but not limited to, ion exchange, alumina, aluminium stearate, lechitin, serum protein (for example, human serum albumin), buffer materials (for example, various phosphate, glycine, sorbic acid, potassium sorbate, partial glyceride mixture of vegetable saturated fatty acids), water, a salt or an electrolyte (for example, protamyne sulfate, disodium hydrogenphosphate, potassium hydrogenphosphate, sodium chloride, and a zinc salt), colloidal silica, magnesium trisilicate, polyvinyl pyrrolidone, cellulose based substrate, polyethylene glycol, sodium carboxylmethylcellulose, polyacrlyate, wax, polyethylene glycol or lanoline. The composition of the present invention may further comprises a lubricant, a wetting agent, an emulsifier, a suspending agent, or a preservative, in addition to the components above.

In one aspect, the composition according to the present invention may be prepared in a form of water soluble solution for the parenteral administration. Preferably, Hank s solution, Ringer s solution or a buffer solution such as a physically buffered saline may be used. To the water soluble suspension for injection may be added a substrate that may increase the viscosity of suspension, such as sodium carboxymethylcellulose, sorbitol or dextran.

Another preferable aspect of the present composition may be in a form of sterile injection formulation in an aqueous or oil suspension. Such suspension may be formulated using a suitable dispersing agent or wetting agent (for example, Tween 80), according to the known technique in this field. The sterile injection formulation may be a sterile injection solution or suspension (for example, a solution in 1,3-butandiol) in a non-toxic, parenterally acceptable diluent or solvent. The usable vehicle and solvent includes mannitol, water, Ringer's solution and an isotonic sodium chloride solution. In addition, sterile nonvolatile oil is usually used as a solvent or a suspending medium. For this purpose any of less irritable nonvolatile oil including synthetic mono or diglyceride may be used.

The present invention also relates to a method for using a contrast composition which comprises the steps of:

administrating the contrast composition according to the present invention to an organism or a specimen; and

sensing signals emitted by the magnetic nanocomposite from the organism or the specimen to obtain images.

The present invention also relates to a method for diagnosing disease which comprises the steps of:

administrating a composition for diagnosis according to the present invention to an organism or a specimen; and

sensing signals emitted by the magnetic nanocomposite from the organism or the specimen to obtain images.

The present invention also relates to a method for simultaneously diagnosing and treating disease which comprises the steps of:

administrating the pharmaceutical composition according to the present invention to an organism or a specimen; and

sensing signals emitted by the magnetic nanocomposite from the organism or the specimen to obtain images.

The term “specimen” used above refers to a tissue or a cell separated from the subject to be diagnosed. In the step for injecting the contrast composition to an organism or a specimen, the contrast composition may be administrated by routes usually used in the pharmaceutical field, and preferably the parenteral administration, for example, the intravenous, intraperitoneal, intramuscular, subcutaneous or topical route.

In the method for using it, the signals emitted by magnetic nanocomposite may be sensed by various apparatuses using the magnetic field, and more preferably, Magnetic Resonance Imaging (MRI) Apparatus.

Magnetic Resonance Imaging Apparatus refers to an apparatus for imaging signals transformed from the emitting energy of an atomic nucleus such as hydrogen through computer processing, wherein the emitting energy is obtained by putting an organism in a powerful magnetic field, irradiating a radio wave with particular frequency on the organism, and stopping the radio wave after an atomic nucleus, such as hydrogen, present in a tissue of the organism absorbs energy and ends up in the upper energy state. The magnetic field or the radio wave is not interfered with bones. Thus, a clear three-dimensional tomographic imaging may be obtained at longitudinal, transverse, an optional angle about tumor of bone surroundings, brain or bone marrow. In particular, the magnetic resonance imaging apparatus is preferably T2 spin-spin relaxation magnetic resonance imaging apparatus.

The present invention relates to a method for separating a target substance by binding magnetic nanocomposite to the target substance and applying a magnetic field to conjugates of the magnetic nanocomposite and the target substance, characterized in that nanoparticle is covered with an amphiphilic compound having one or more of hydrophobic domains and one or more hydrophilic domains, and one or more binding parts for a hydrophilic active ingredient are bound to a tissue-specific binding ingredient.

In the method for separating according to the present invention, the preferable example of a target substance refers to a biological molecule, more specifically, includes, but not limited to, a cell, a protein, an antigen, a peptide, DNA, RNA, or a virus.

The magnetic nanocomposite formed according to the present invention may be used in a nanoprobe for separation, diagnosis, treatment, etc. of a biological molecule, and a drug or gene delivery system, and the like.

A representative example of biological diagnosis using magnetic nanocomposite includes molecular magnetic resonance imaging diagnosis or magnetic relaxation sensor. The magnetic nanocomposite shows much better T2 contrasting effect, as its size is increased. Using such property, the magnetic nanocomposite may be used in a sensor for detecting biological molecules. That is, particular biological molecules lead to aggregation of the magnetic nanocomposite, whereby the T2 magnetic resonance imaging effect is increased. The biological molecule is detected using this difference.

In addition, the magnetic nanocomposite according to the present invention can constitute a diagnosing material for Giant magnetic resistance (GMR) sensor. The magnetic nanocomposite may show more excellent magnetic characteristic, better stability of colloid in a water solution and lower non-selective binding than that of conventional beads with micrometer (10⁻⁶ m) size (U.S. Pat. No. 6,452,763 B1; U.S. Pat. No. 6,940,277 B2; U.S. Pat. No. 6,944,939 B2; US 2003/0133232 A1), and thus have the possibility to improve the detecting limit of conventional GMR sensor.

The magnetic nanocomposite may be also used in separation and detection using magnetic micro fluid sensors, delivery of drugs or genes, and magnetic hyperthermia.

Meanwhile, the magnetic nanocomposite according to the present invention may be used in dual- or multi-diagnostic probe, combining with other diagnostic probes. For example, when a water soluble magnetic nanocomposite is combined with a diagnostic probe of T1 magnetic resonance imaging, simultaneous diagnosis for T2 magnetic resonance imaging and T1 magnetic resonance imaging can be performed. When the nanocomposite is combined with an optical diagnostic probe, magnetic resonance imaging and optical imaging can be simultaneously performed. When the nanocomposite is combined with CT diagnostic probe, magnetic resonance imaging and CT diagnosis can be simultaneously performed. In addition, when the nanocomposite is combined with radioisotopes, magnetic resonance imaging, PET, SPECT can be simultaneously performed.

The diagnostic probe of T1 magnetic resonance imaging comprises a Gd compound, a Mn compound, and the like; the optical diagnostic probe comprises an organic fluorescent dye, a quantom dot, or a dye labeled inorganic support (for example, SiO₂, Al₂O₃); the CT diagnostic probe comprises a I (iodine) compound, a gold nanoparticle; and the radioisotope comprises In, Tc, F and the like.

### Advantageous Effects

The magnetic nanocomposite according to the present invention, covered with the amphiphilic compound having hydrophobic domains and a hydrophilic domains may be used in a contrast agent for high sensitive MRI, an intelligent contrast agent for diagnosing cancer by binding to the binding parts materials that may specifically be bound to tumor markers, a drug delivery system for diagnosis and treatment of cancer by polymerizing or enclosing a drug in the hydrophobic domains, and a formulation for separating cells and proteins using magnetism by binding an antibody or a protein specific to surface antigens of functional cells, stem cells or cancer cells thereto.

### DESCRIPTION OF DRAWINGS

FIG. 1 is a schematic diagram which depicts applications of the magnetic nanocomposite according to the present invention.

FIG. 2 is a schematic diagram which depicts the method for preparing a magnetic nanocomposite using an amphiphilic polymer, according to one embodiment of the present invention.

FIGS. 3a-h are concept diagrams of the emulsion type or suspension type magnetic nanocomposite according to one embodiment of the present invention.

FIGS. 4a-c are transmission electron microphotographs of the magnetic nanoparticle using a saturated fatty acid, according to one embodiment of the present invention and a graph which depicts its magnetic property.

FIGS. 5a-c are transmission electron microphotographs of the magnetic nanoparticle using a unsaturated fatty acid, according to another embodiment of the present invention and a graph which depicts its magnetic property.

FIG. 6 is a schematic diagram which depicts the method for preparing a magnetic nanocomposite using a fatty acid amphiphilic polymer, according to one embodiment of the present invention.

FIG. 7 is a graph which depicts the infrared spectrometry (FT-IR) results of the magnetic nanoparticles and magnetic nanocomposite according to one embodiment of the present invention.

FIG. 8 is a graph which depicts the Proton Nuclear Magnetic Resonance (¹H-NMR) result of the fatty acid amphiphilic compound according to one embodiment of the present invention.

FIG. 9 is a schematic diagram which depicts the polymerizing process of the amphiphilic polymer whose binding part for a hydrophilic active ingredient is substituted with a carboxyl group by binding the active ingredient of a polymer, according to one embodiment of the present invention.

FIG. 10 is a graph which depicts the ¹H-NMR result of the biodegradable amphiphilic compound whose binding part is substituted with a carboxyl group, according to the present invention.

FIG. 11 is a graph which depicts the IR spectrometry result of the biodegradable amphiphilic compound whose binding part is substituted with a carboxyl group, according to the present invention.

FIG. 12 is a schematic diagram which depicts the polymerizing process of biodegradable amphiphilic polymer whose binding part for a hydrophilic active ingredient is substituted with a carboxyl group via active ingredient of a polymer, according to the present invention.

FIG. 13 is a graph which depicts the IR spectrometry (FT-IR) result of the biodegradable amphiphilic compound according to one embodiment of the present invention.

FIG. 14 are graphs which depicts the ¹H-NMR result of the amphiphilic polymer according to another preparation example of the present invention.

FIGS. 15a and b are the synthesizing process of biodegradable amphiphilic polymer substituted with a binding part for a hydrophilic active ingredient.

FIGS. 16a and b are electron microphotographs of the emulsion type magnetic nanoparticles using the nanoparticles and a biodegradable amphiphilic polymer, according to one embodiment of the present invention and a graph which depicts their size distribution.

FIGS. 17a and b are electron microphotographs of the suspension type magnetic nanoparticles using the nanoparticles and a biodegradable amphiphilic polymer, according to one embodiment of the present invention and a graph which depicts their size distribution.

FIGS. 18a and b are electron microphotographs of the emulsion type magnetic nanoparticles using the nanoparticles and a fatty acid amphiphilic polymer, according to one embodiment of the present invention and a graph which depicts their size distribution.

FIG. 19 is a graph which depicts the result of hysteresis loop in the emulsion type magnetic nanocomposite using a fatty acid amphiphilic compound, according to one embodiment of the present invention.

FIG. 20 are electron microphotographs showing the state that the magnetic nanoparticles according to the present invention are enclosed by carboxylpolyethyleneglycol-polylactide-co-glycolide and a graph which depicts their size distribution.

FIG. 21 is a graph which depicts the ratio by weight in the state that the magnetic nanoparticles according to the present invention are enclosed by carboxylpolyethyleneglycol-polylactide-co-glycolide.

FIG. 22 is hysteresis loops of magnetic nanoparticles and magnetic nanocomposite, according to the present invention.

FIGS. 23a and b are an electron microphotographs of the magnetic nanocomposite prepared by the suspension method according to the present invention and a graph which depicts their size distribution by a dynamic laser light scattering method.

FIG. 24 is a thermogravimetric analysis graph of the magnetic nanoparticles prepared by the suspension method according to the present invention.

FIG. 25 is a are transmission electron microphotograph of the water soluble magnetic nanocomposite according to one embodiment of the present invention and a graph which depicts the result of dynamic laser light scattering method.

FIG. 26 is a graph which depicts the IR spectrometry result of the water soluble magnetic nanocomposite according to one embodiment of the present invention.

FIGS. 27a and b are an electron microphotographs of the magnetic nanocomposite prepared by the suspension method according to the present invention and their size distribution view by a light scattering method.

FIG. 28 is a thermogravimetric analysis graph of the magnetic nanoparticles prepared by the suspension method according to the present invention.

FIG. 29 is an electron microphotograph of the nanocomposite prepared by the emulsion method of the present invention, in which MnFe₂O₄ is enclosed by polylactide-co-glycolide-polyethyleneglycol, and a size distribution view by a light scattering method.

FIG. 30 is a result of the ratio by weight obtained through thermogravimetric analysis in the state that MnFe₂O₄ prepared according to the present invention is enclosed by polylactide-co-glycolide-polyethyleneglycol, and its hysteresis loops.

FIG. 31 is photographs showing the arrangement of the magnetic nanocomposite by an external magnetic field, in which a fluorescent dye is enclosed, according to the present invention.

FIGS. 32a-c show solubility of organic nanoparticles in an organic solvent and solubility of water soluble magnetic nanocomposite, using a biodegradable amphiphilic compound, in a water solution, according to one embodiment of the present invention.

FIGS. 33a and b show solubility of organic nanoparticles in an organic solvent, solubility of water soluble magnetic nanocomposite, using a fatty acid amphiphilic compound, in a water solution, and a response appearance in an external magnetic field, according to one embodiment of the present invention.

FIGS. 34a and b are graphs which depict salt concentrations of a water soluble magnetic nanocomposite using a fatty acid amphiphilic compound according to one embodiment of the present invention and the stability test results of them with pH.

FIG. 35 is a photograph showing the particle stability of the water soluble magnetic nanocomposite with pH, according to one embodiment of the present invention and a graph of size change with pH.

FIG. 36 is a photograph showing the particle stability of the water soluble magnetic nanocomposite with salt concentrations, according to one embodiment of the present invention and a graph of size change with salt concentration.

FIG. 37 is a graph which depicts the change of MRI signals (T2) with concentrations of the water soluble magnetic nanocomposite using a biodegradable amphiphilic compound, according to one embodiment of the present invention.

FIG. 38 is a graph which depicts the change of MRI signals (T2) with concentrations of the water soluble magnetic nanocomposite using a fatty acid amphiphilic compound, according to another embodiment of the present invention.

FIG. 39 is photographs that MRI is identified with concentrations of solutions in which magnetic nanoparticles prepared by the suspension method according to the present invention are dispersed and a graph of R2 value change.

FIG. 40 is a solution MRI photograph of the water soluble magnetic nanocomposite according to one embodiment of the present invention.

FIG. 41 is a graph which depicts T2 value of MRI of the water soluble magnetic nanocomposite with Fe concentrations, according to one embodiment of the present invention.

FIG. 42 is photographs that MRI is identified with concentrations of solutions dispersing magnetic nanoparticles whose hydrophilic end group is substituted with a succinimidyl group, according to the present invention, and a graph of T2 value change with concentrations.

FIG. 43 is photographs that MRI is identified with concentrations of solutions in which magnetic nanoparticles prepared by the suspension method according to the present invention are dispersed, and a graph of T2 value change with concentrations.

FIG. 44 is a graph which depicts fluorescence intensity by Fluorescence Activated Cell Sorter (FACS) of the cell reacted with the intelligent contrast agent for MRI according to one embodiment of the present invention.

FIG. 45 is MRI photographs of the positive cells reacted with the intelligent contrast agent for MRI according to one embodiment of the present invention.

FIG. 46 is a result of analyzing cell specificity of Herceptin-magnetic nanocomposite according to the present invention.

FIG. 47 is a view identifying affinity of Herceptin-magnetic nanocomposite, in which MnFe₂O₄ is enclosed, according to the present invention, to a cancer cell by flow cytometry.

FIG. 48 is a view identifying by flow cytometry to estimate the degree of binding Herceptin-magnetic nanocomposite and cells, according to the present invention.

FIG. 49 is a photograph obtained by MRI, after the emulsion type Herceptin-magnetic nanocomposite prepared according to another embodiment of the present invention is reacted with a target cell line (MDA-MB-231, NIH3T6.7 cell line), and a comparative graph of T2 value.

FIG. 50 is a photograph obtained by MRI, after the suspension type Herceptin-magnetic nanocomposite prepared according to another embodiment of the present invention is reacted with a target cell line (MDA-MB-231, NIH3T6.7 cell line), and a comparative graph of T2 value.

FIG. 51 is a graph of drug release behavior in the emulsion type magnetic nanocomposite according to one embodiment of the present invention.

FIG. 52 is a graph of drug release behavior in the emulsion type magnetic nanocomposite according to another embodiment of the present invention.

FIG. 53 is a graph of drug release behavior in the suspension type magnetic nanocomposite according to another embodiment of the present invention.

FIG. 54 is a view identifying affinity of the magnetic nanocomposite, in which MnFe₂O₄ is enclosed, according to the present invention, to a target cell by flow cytometry.

FIG. 55 is photographs identifying an appearance that the target cells attached with magnetic nanocomposite, in which MnFe₂O₄ is enclosed, according to the present invention, are moved toward one side surface of a wall by an external magnetic field.

FIGS. 56 to 58 are graphs which depict the cytotoxic test results of the water soluble magnetic nanocomposite according to one embodiment of the present invention.

FIGS. 59, 60 and 62 are MRIs of animal models scanned using the water soluble magnetic nanocomposite according to one embodiment of the present invention.

FIGS. 61 and 63 are graphs of R2 value change with injection time period of the intelligent contrast agent for MRI according to one embodiment of the present invention.

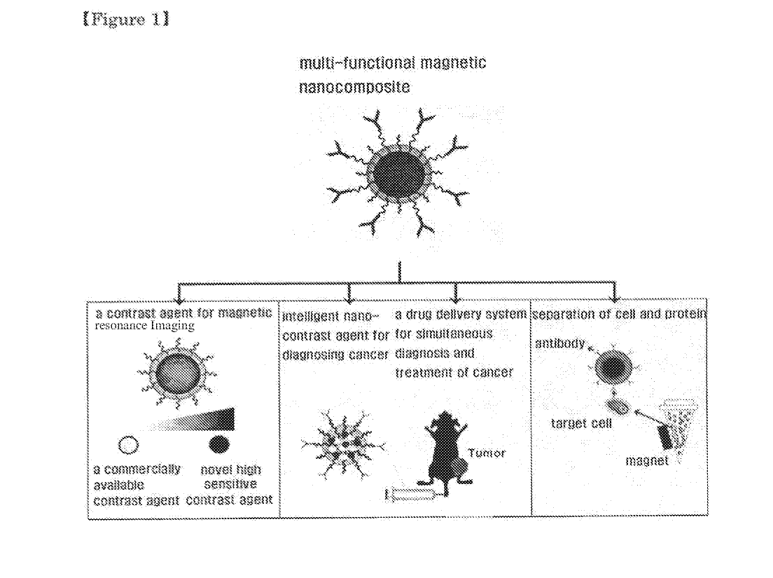

### BEST MODE

The present invention is described by examples in more detail below. However, the examples below are intended to illustrate the present invention, do not limit the present invention in any manner.

### Preparation Example 1

### Preparation of High Sensitive Magnetic Nanoparticles Using a Saturated Fatty Acid

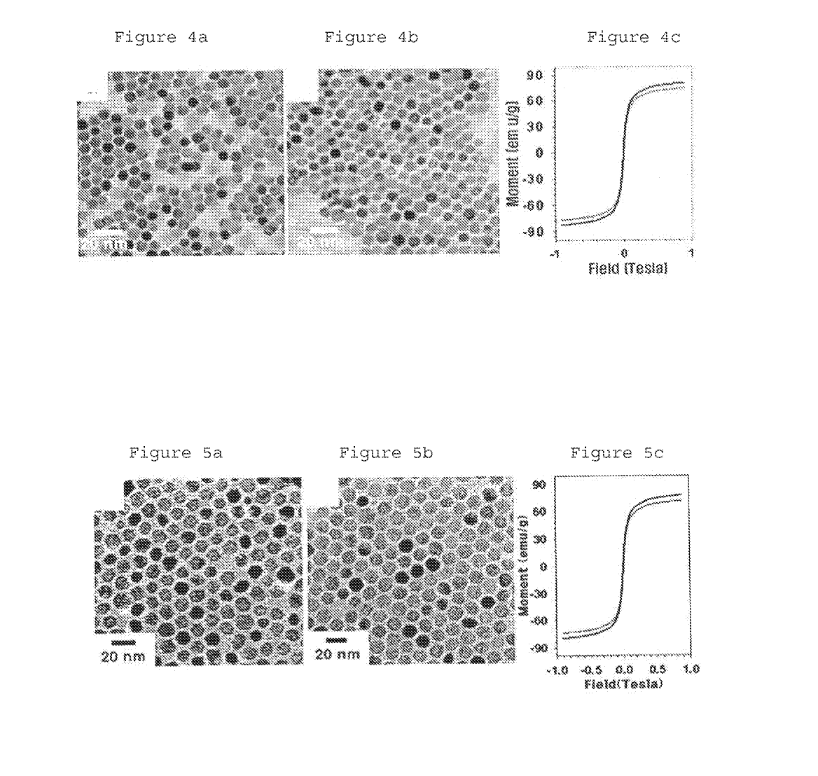

Dodecanoic acid (0.6 mol) and dodecylamine (0.6 mol) in a benzylether solvent and iron triacetylacetonate (Aldrich) were thermolyzed at 290° C. for 30 minutes to synthesize 6 nm magnetite (Fe₃O₄). The benzylether solution including dodecanoic acid (0.2 mol), dodecylamine (0.1 mol), said 6 nm iron oxide nanoparticles (10 mg/ml) and iron triacetylacetonate was heated at 290° C. for 30 minutes to prepare 12 nm iron oxide nanoparticles. To the reaction above was added manganese II acetylacetonate to prepare manganese ferrite (MnFe₂O₄). Transmission electron microphotographs of the prepared magnetite and manganese ferrite were depicted in FIGS. 4a and 4b, respectively. The magnetic property of magnetite and manganese ferrite was measured using VSM. The measurements were represented by a dotted line and a solid line, respectively and depicted in FIG. 4c.

### Preparation Example 2

### Preparation of High Sensitive Magnetic Nanoparticles Using an Unsaturated Fatty Acid

Oleic acid (0.6 mol) and olecylamine (0.6 mol) in a benzylether solvent and iron triacetylacetonate (Aldrich) were thermolyzed at 290° C. for 30 minutes to synthesize 6 nm magnetite (Fe₃O₄). The benzylether solution including oleic acid (0.2 mol), oleylamine (0.1 mol), said 6 nm iron oxide nanoparticles (10 mg/ml) and iron triacetylacetonate was heated at 290° C. for 30 minutes to prepare 12 nm iron oxide nanoparticles. To the reaction above was added manganese II acetylacetonate to prepare manganese ferrite (MnFe₂O₄). Transmission electron microphotographs of the prepared magnetite and manganese ferrite were depicted in FIGS. 5a and 5b, respectively. The magnetic property of magnetite and manganese ferrite was measured using VSM. The measurements were represented by a dotted line and a solid line, respectively and depicted in FIG. 5c.

### Preparation Example 3

### Polymerization of a Biodegradable Amphiphilic Polymer, Monomethoxy-Polyethyleneglycol-Polylactide-Co-Glycolide

Moisture was removed from 2 g of monomethoxypolyethyleneglycol (MPEG, molecular weight 5000) under reduced pressure. 2.0 mg of stannous octoate as a catalyst was added to absolute toluene, followed by reducing pressure at 100° C. for 20 to 30 minutes. 1.15 g of D,L-lactide and 0.93 g of glycolide were added to the reaction and polymerized at 140° C. for 12 h. To 5 ml of chloroform, the resulting block copolymer was added to be dissolved. An excess of diethylether was portionwise dropped on the solution to obtain a precipitate, which was subsequently filtered, washed with diethylether and dried at 50° C. under reduced pressure to obtain a block copolymer of monomethoxypolyethyleneglycol-polylactide-co-glycolide (Yield 72.5%, including a loss amount).

A variety of double block copolymers were prepared using components described in Table 5 below, by the same method above. Output and yield of the resulting double block copolymers are as follows:

TABLE 5

| | Amount of Reactants | | | | |
|---|---|---|---|---|---|
| Kind of | | D,L- | | stannous- | Yield of |
| Block | MPEG | lactide | Glycolide | octoate | Copolymer |
| Copolymer | (g) | (g) | (g) | (mg) | (%) |
| MPEG(5000)- | 2 | 0.2306 | 0.1856 | 50 | 69.3 |
| PLGA(1000) | | | | | |
| MPEG(5000)- | 2 | 1.1530 | 0.9280 | 50 | 68.6 |
| PLGA(5000) | | | | | |
| MPEG(2000)- | 2 | 0.5765 | 0.4640 | 50 | 71.3 |
| PLGA(1000) | | | | | |
| MPEG(2000)- | 2 | 3.4591 | 2.7840 | 50 | 70.1 |
| PLGA(5000) | | | | | |

The resulting block copolymers were identified by ¹H-NMR, with representing a peak of polyethyleneglycol adjacent to 3.6 ppm and peak of polylactide-co-glycolide adjacent to 4.9 and 1.6 ppm. Relative molecular weights and molecular weight distributions of the resulting block copolymers by gel permeation chromatography (GPC) are set forth in Table 6 below.

TABLE 6

| | | |
|---|---|---|
| Kind of Block | NMR | GPC |

| Copolymer | Mₙₐ | M_{nb} | M_{w}/Mₙ |
|---|---|---|---|
| MPEG(5000)- | 6270 | 6710 | 1.19 |
| PLGA(1000) | | | |
| MPEG(5000)- | 10350 | 12470 | 1.21 |
| PLGA(5000) | | | |
| MPEG(2000)- | 3280 | 3720 | 1.15 |
| PLGA(1000) | | | |
| MPEG(2000)- | 9510 | 9980 | 1.11 |
| PLGA(5000) | | | |

ₐNMR
_{b}Gel permeration chromatography
Mₙ: Number Average Molecular Weight
M_{w}: Weight Average Molecular Weight

### Preparation Example 4

### Polymerization of a Fatty Acid Amphiphilic Compound, Monomethoxypolyethylene-Glycol-Dodecanoic Acid

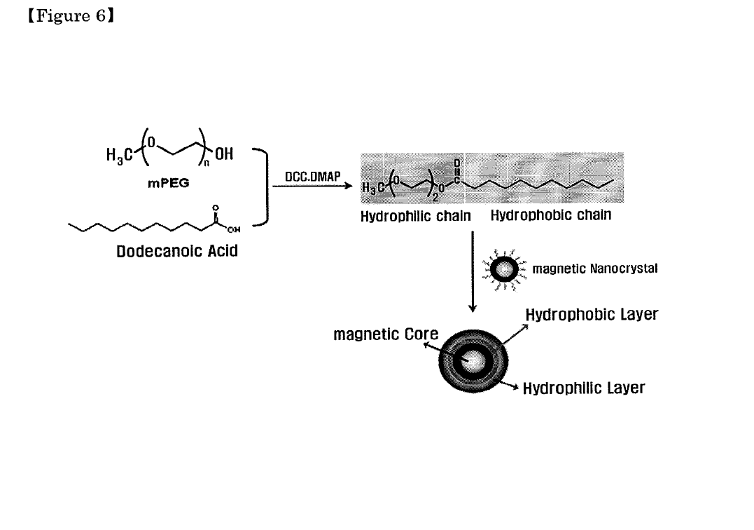

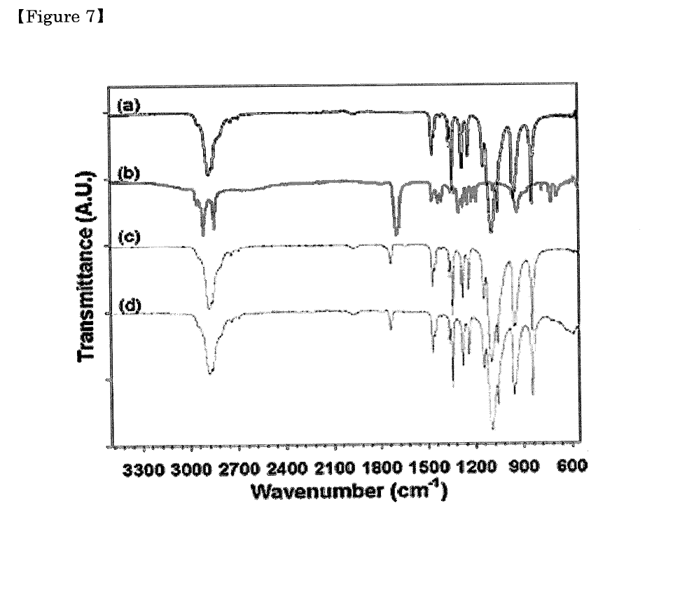

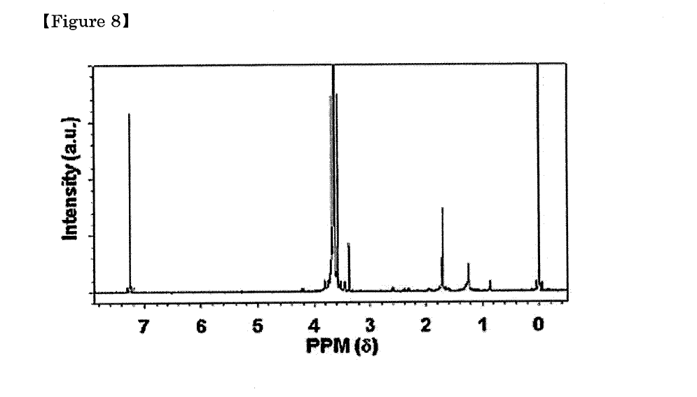

The process of polymerizing a fatty acid amphiphilic compound, monomethoxypolyethyleneglycol-dodecanoic acid was depicted in FIG. 6. 5 g of monomethoxypolyethyleneglycol (MPEG) with an average molecular weight of 5,000 and 0.6 g of dodecanoic acid (DA) were dissolved in methylene chloride, and then 0.91 g of 1,3-dicyclohexylcarbodiimide and 0.37 g of 4-dimethylaminopyridine were added thereto to proceed the reaction. After 24 h, the obtained by-product was filtered off and an excess of cold diethylether was added. The resulting precipitate was filtered, washed with diethylether, and dried under reduced pressure to prepare an amphiphilic polymer of monomethoxypolyethyleneglycol-dodecanoic acid (MPEG-DA) (Yield 92.5%). The structure of polymer was identified by FT-IR and ¹H-NMR, and the results were depicted in FIGS. 7 and 8, respectively. In FIG. 7, the spectrums of water soluble magnetic nanocomposite are represented, using (a) monomethoxypolyethyleneglycol, (b) dodecanoic acid, (c) monomethoxy-polyethyleneglycol-dodecanoic acid and (d) monomethoxypolyethyleneglycol-dodecanoic acid. As shown in FIG. 7, a peak of carboxylic acid (—COOH) in dodecanoic acid was identified at 1695 cm⁻¹ and a peak of an ester bond, being binding part of dodecanoic acid and polyethylene glycol, was identified at 1734 cm⁻¹ by IR Spectroscopy. As shown in FIG. 8, using ¹H-NMR, a peak of —CH₂CH₂O— in monomethoxypolyethylene glycol was identified at 3.62 ppm, and a peak of dodecanoic acid was identified at 1.27 ppm.

### Preparation Example 5

### Synthesis of a Biodegradable Amphophilic Polymer that a Binding Part for a Hydrophilic Active Ingredient is Substituted with a Carboxyl Group

A. Synthesis of a Biodegradable Amphiphilic Polymer that a Binding Part for a Hydrophilic Active Ingredient is Substituted with a Carboxyl Group, by Combining an Active Ingredient of Polymer

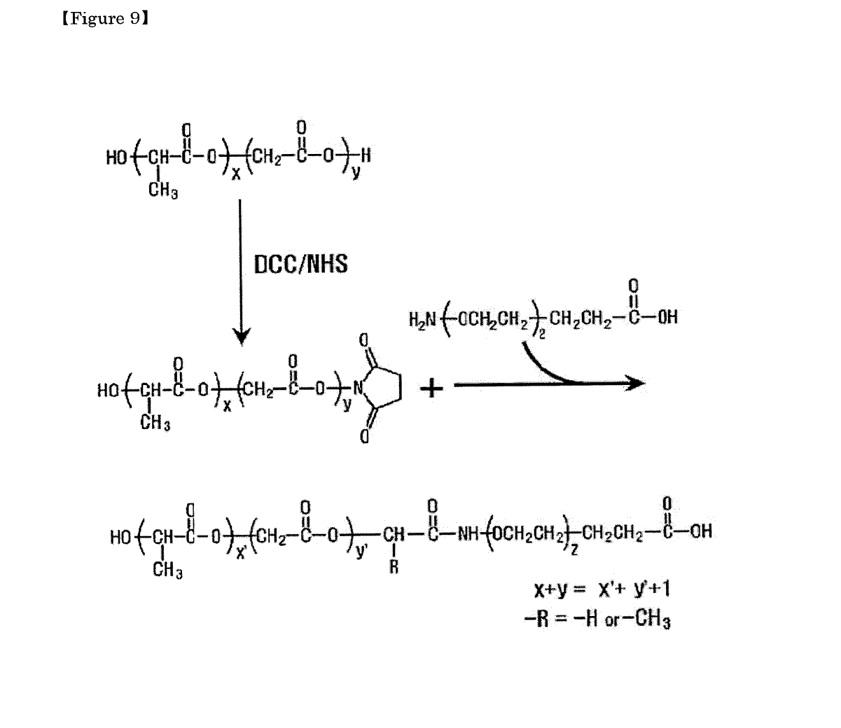

The process of synthesizing biodegradable amphiphilic polymer that a binding part for a hydrophilic active ingredient was substituted with a carboxyl group, by combining an active ingredient of polymer was depicted in FIG. 9. 0.05 mol of polylactide-co-glycolide, 0.2 mol of N-hydroxysuccinimide (NHS) and 1,3-dicyclohexylcarbodiimide (DCC) were dissolved in methylene chloride, and then reacted at room temperature for 24 h under nitrogen atmosphere. The reactant was filtered through a filter and dropped on cold diethylether to be precipitated. This precipitate was washed several times with diethylether, and then stored in vacuum.

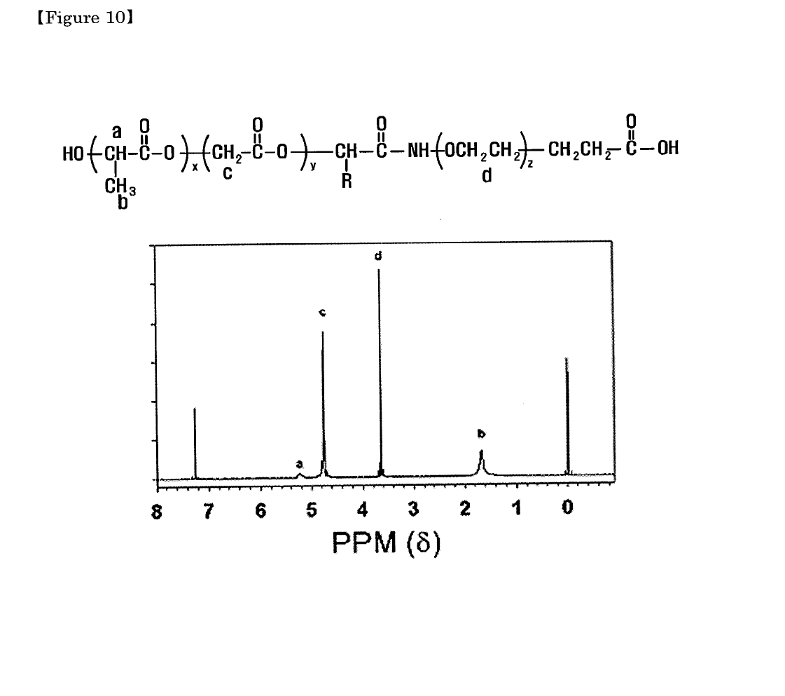

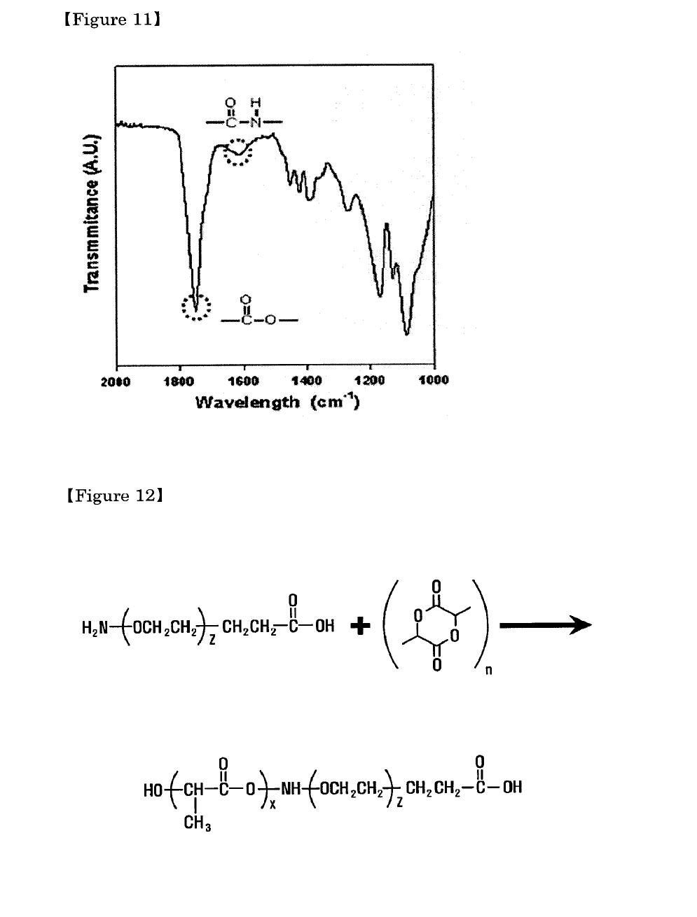

0.01 mol of the polymer activated by the above method was taken and dissolved in 8 ml of methylene chloride. 0.01 mol of polyethylene glycol, both ends whose terminal functional groups were substituted with an amine group and a carboxyl group, was taken and dissolved in 2 ml of methylene chloride, and was reacted, with the solution dropped portionwise. The reaction was subjected at room temperature for 12 h under nitrogen atmosphere. The reactant was washed and stored by the method as mentioned above. The structure of the synthesized polymer was analyzed by ¹H-NMR and FT-IR, and the results were depicted in FIGS. 10 and 11.

B. Polymerization of a Biodegradable Amphophilic Polymer that a Binding Part for a Hydrophilic Active Ingredient is Substituted with a Carboxyl Group Through an Active Ingredient of Hydrophilic Polymer

The process of polymerizing a biodegradable amphophilic polymer that a binding part for a hydrophilic active ingredient was substituted with a carboxyl group through an active ingredient of hydrophilic polymer was depicted in FIG. 12. Moisture in 0.2 g of polyethylene glycol (molecular weight of 3400), both ends whose terminal functional groups were substituted with an amine group and a carboxyl group, was removed under reduced pressure. 20 mg of stannous octoate as a catalyst was added to absolute toluene, followed by reducing pressure at 100° C. for 20 to 30 minutes. 0.119 g of D,L-lactide was added to the reactant and polymerized at 140° C. for 12 h. To 5 ml of chloroform, the resulting block copolymer was added to be dissolved. An excess of diethylether was portionwise dropped on the solution to obtain a precipitate, which was subsequently filtered, washed with diethylether and dried at 50° C. under reduced pressure overnight to obtain a block copolymer of monomethoxypolyethyleneglycol-polylactide-co-glycolide (Yield 87.2%).

### Preparation Example 6

### Synthesis of Amphiphilic Polymer that a Binding Part for a Hydrophilic Active Ingredient of a Commercially Available Surfactant is Substituted with a Carboxyl Group

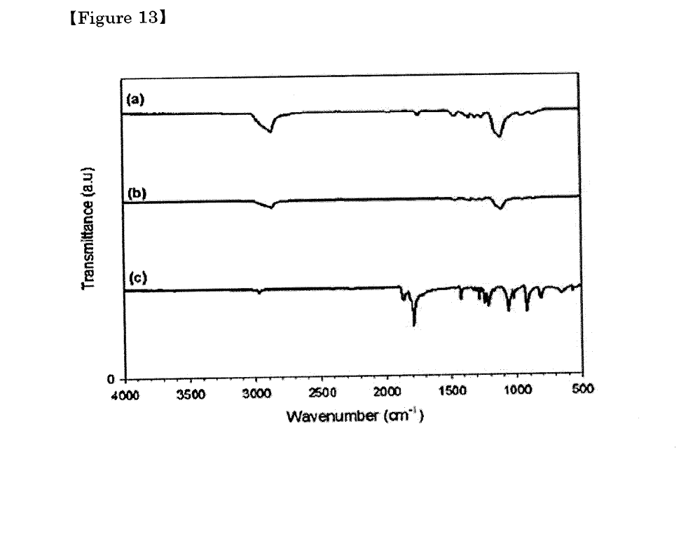

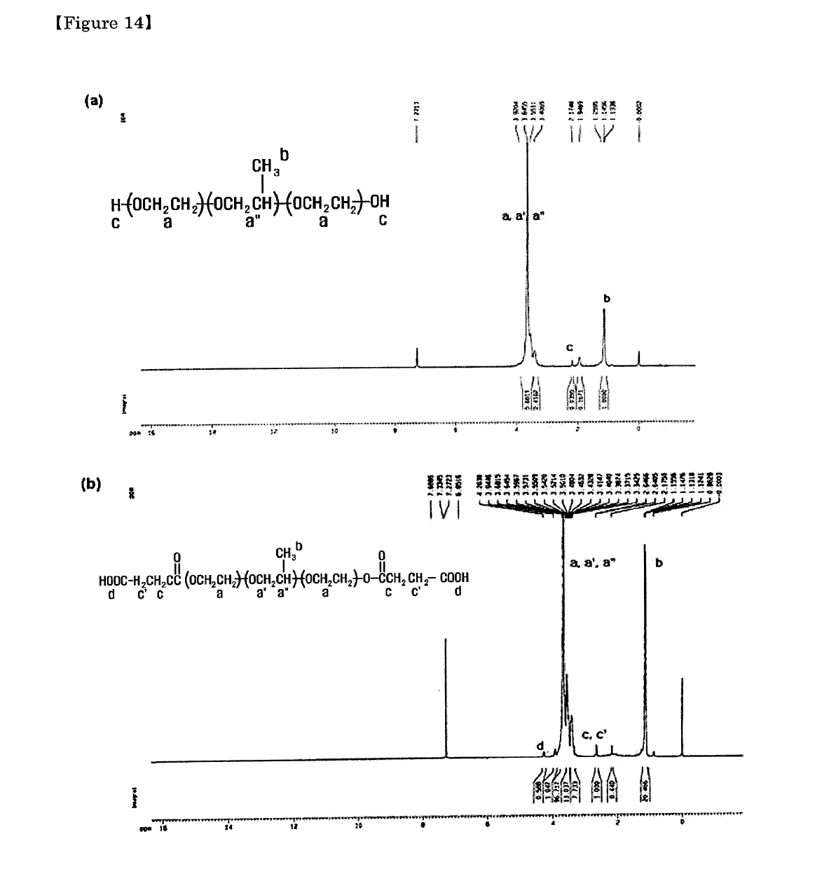

A Pluronic based nonionic commercially available surfactant has a form of polyethyleneoxide-polypropyleneoxide-polyethyleneoxide (PEO-PPO-PEO, hydrophilic-hydrophobid-hydrophilic). The terminal hydroxyl group (—OH) of this surfactant was substituted with a carboxyl group to which a ligand such as an antibody may be bound. 30 g of Pluronic F-127, 476.5 mg of succinic anhydride as a carboxyl group substituent, 290.9 mg of 4-dimethylaminopyridine as a catalyst, and 331.9 μl of triethylamine were dissolved in 500 ml of 1,4-dioxane as a solvent, and reacted for 24 h at room temperature. After completing the reaction, the solvent was removed by lyophilization, followed by adding carbon tetrachloride and filtering through a filter to remove the unreacted succinic anhydride. In order to remove the remaining impurities, the filtered reactant was dropped on cold diethylether to be precipitated. This precipitate was washed several times with diethylether and stored. Pluronic F-127 that a binding part for a hydrophilic active ingredient was substituted with a carboxyl group was identified by analyzing IR spectroscopy and ¹H-ISTMR. The results were depicted in FIGS. 13 and 14, respectively. In FIG. 13, line (a) represents a peak of Pluronic F-127 that a binding part for a hydrophilic active ingredient was substituted with a carboxyl group, FIG. 13, line (b) represents a peak of Pluronic F-127 and FIG. 13, line (c) represents a peak of succinic anhydride. Also, in FIG. 14, graph (a) is the ¹H-NMR result of Pluronic F-127 before substituting a binding part for a hydrophilic active ingredient with a carboxyl group, according to another Preparation Example of the present invention, FIG. 14, graph (b) is the ¹H-NMR result of Pluronic F-127 substituted with a carboxyl group.

### Preparation Example 7

### Synthesis of Biodegradable Amphiphilic Polymer that a Binding Part for a Hydrophilic Active Ingredient is Substituted with a Succinimidyl Group

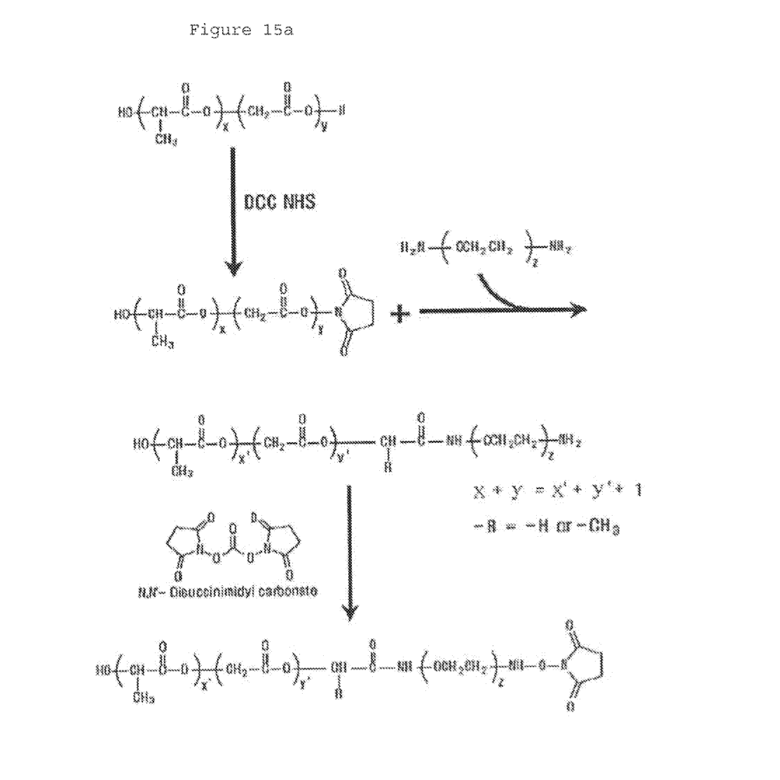

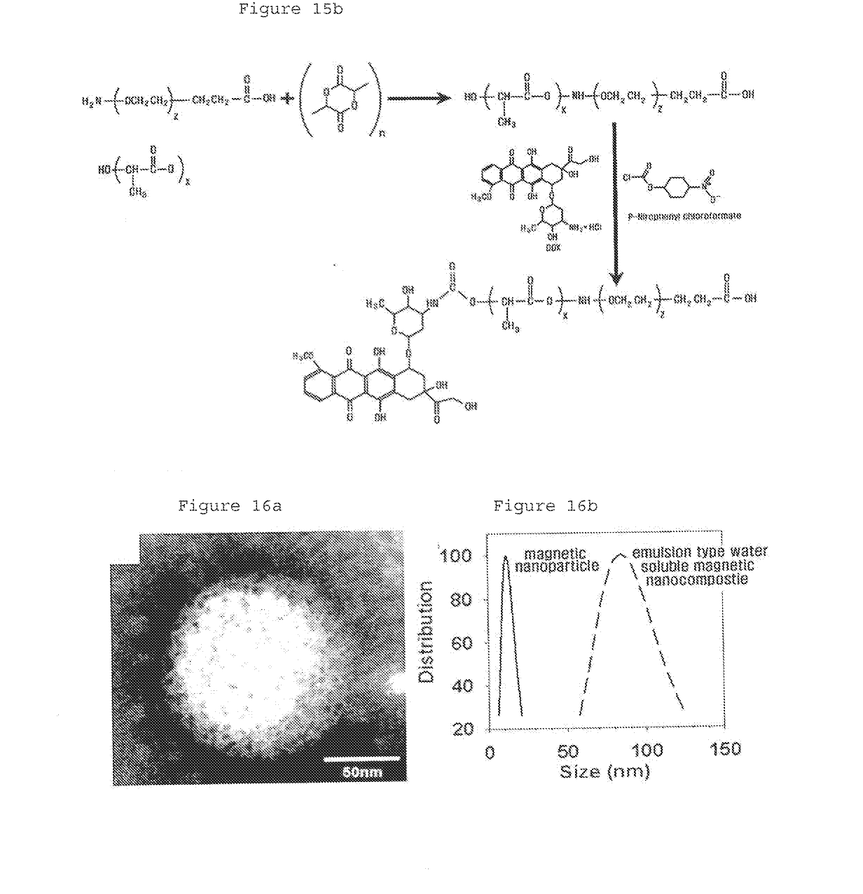

A biodegradable amphiphilic polymer that a binding part for a hydrophilic active ingredient was substituted with a succinimidyl group was synthesized through the process shown in FIG. 15a. 0.05 mol of polylactide-co-glycolide, 0.4 mol of N-hydroxysuccinimide (NHS) and 1,3-dicyclohexylcarbodiimide were dissolved in methylene chloride, and then reacted at room temperature for 24 h under nitrogen atmosphere. The reactant was filtered through a filter and dropped on cold diethylether to be precipitated. This precipitate was washed several times with diethylether, and then stored in vacuum. 0.01 mol of the polymer activated by the above method was taken and dissolved in 8 ml of methylene chloride. 0.05 mol of polyethylene glycol (molecular weight 3,400), both ends whose terminal functional groups were substituted with amine groups, was taken and dissolved in 2 ml of methylene chloride, and was reacted, with the solution dropped portionwise. The reaction was subjected at room temperature for 12 h under nitrogen atmosphere. The reactant was washed and stored by the method as mentioned above. N,N′-Disuccinimidyl carbonate was used in transforming the hydrophilic terminal functional group substituted with a amine group of the biodegradable amphiphilic polymer into a succinimidyl group to which an amine group of an antibody may be bound. 0.01 mol of N,N′-Disuccinimidyl carbonate was taken and dissolved in 4 ml of methylene chloride, 0.05 mol of amphiphilic polymer that a hydrophilic part was substituted with an amine group was dissolved in 1 ml of methylene chloride, and was reacted, with the solution dropped portionwise. The reaction was subjected for 4 h under nitrogen atmosphere. Through gel filtration process (Sephadex G-25), N,N′-Disuccinimidyl carbonate not bound to polymer was removed.

### Preparation Example 8

### Preparation of an Amphiphilic Polymer for Enclosing a Drug in a Magnetic Nanocomposite

The binding of an anticancer agent to a hydrophobic active binding domain of an amphiphilic polymer was subjected through the process as shown in FIG. 15b. The biodegradable amphiphilic polymer that a binding part for a hydrophilic active ingredient was substituted with a carboxyl group, as prepared in Preparation Example 5, B., and p-Nitrophenyl chloroformate were dissolved in absolute methylene chloride. To the solution at 0° C., pyridine was added and reacted at room temperature for 3 h under nitrogen atmosphere. For binding the activated amphiphilic polymer and the anticancer agent, triethylamine was added to dimethylformaldehyde in which doxorubicin (DOX) was dissolved, and reacted at room temperature for 3 h under nitrogen atmosphere. The unreacted DOX and other materials were removed by several times of separations.

### Example 1

### Preparation of an Emulsion Type Magnetic Nanocomposite Using a Biodegradable Amphiphilic Polymer

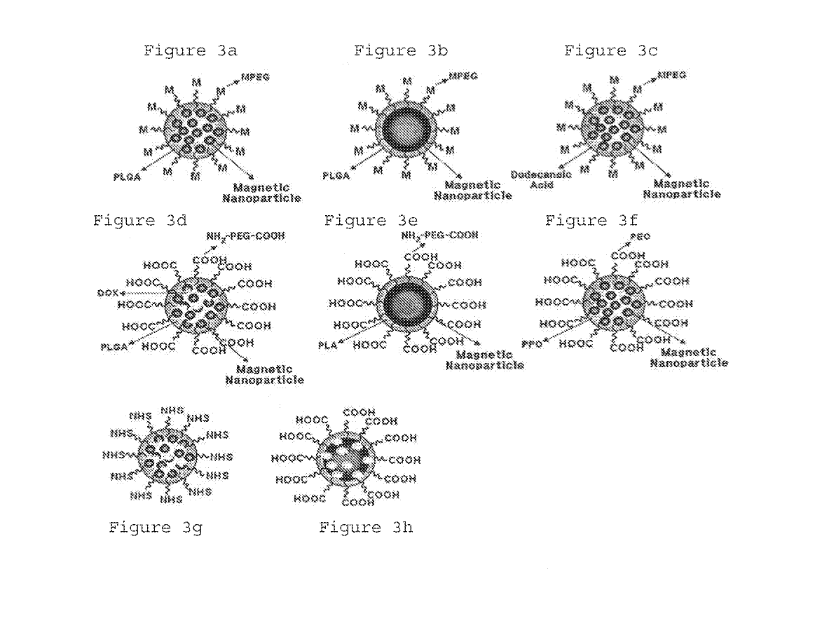

100 mg of Amphiphilic biodegradable polymer, monomethoxypolyethyleneglycol-polylactide-co-glycolide, prepared in Preparation Example 3 above was dissolved in 20 ml of deionized water as an aqueous phase. 20 mg of magnetic nanoparticles prepared in Preparation Example 1 were dissolved in 5 ml of chloroform as an oil phase. The aqueous phase was mixed with the oil phase, and then the mixture was saturated for 10 minutes by ultrasound of 300 W. The resulting emulsion was stirred for 12 h to vaporize the oil phase, and subjected to centrifugation and a gel filtration column (Sephacryl S-300) to prepare the emulsion type magnetic nanocomposite with removed impurities. The schematic diagram of the emulsion type magnetic nanocomposite using the biodegradable amphiphilic polymer, monomethoxypolyethyleneglycol-polylactide-co-glycolide, was depicted in FIG. 3a. The prepared particles were identified by a transmission electron microscope and a dynamic laser light scattering method, and the results were depicted in FIGS. 16a and 16b, respectively.

### Example 2

### Preparation of a Suspension Type Magnetic Nanocomposite Using a Biodegradable Amphiphilic Polymer

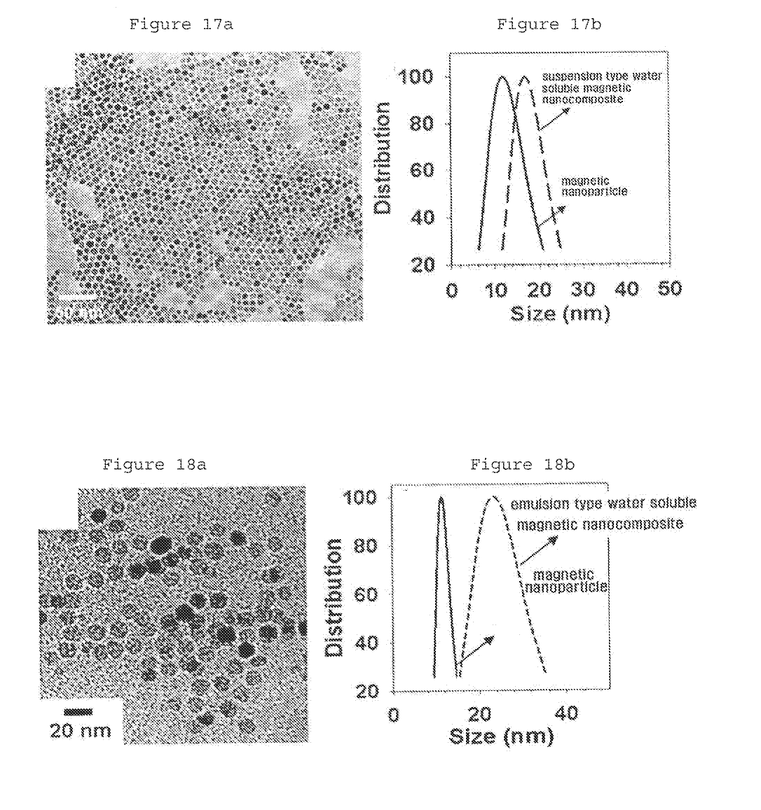

3 mg of Magnetic nanoparticles prepared in Preparation Example 1 above were dispersed in a chloroform solution dissolving 50 mg of the amphiphilic biodegradable polymer (monomethoxypolyethyleneglycol-polylactide-co-glycolide) prepared in Preparation Example 3 above. The dispersion was heated to 40° C., with stirring, to vaporize the solvent, and re-dispersed in 0.5 ml of a phosphate buffered saline (PBS) solution. The solution was heated/stirred at 30° C. for 6 h to complete the suspension. After removing micelles without magnetic particles through centrifugation, the suspension was re-dispersed in 0.5 ml of a PBS solution. The schematic diagram of a suspension type magnetic nanocomposite using the biodegradable amphiphilic polymer, monomethoxypolyethyleneglycol-polylactide-co-glycolide, was depicted in FIG. 3b. The prepared particles were identified by a transmission electron microscope and a dynamic laser light scattering method, and the results were depicted in FIGS. 17a and 17b, respectively.

### Example 3

### Preparation of an Emulsion Type Magnetic Nanocomposite Using a Fatty Acid Amphiphilic Compound

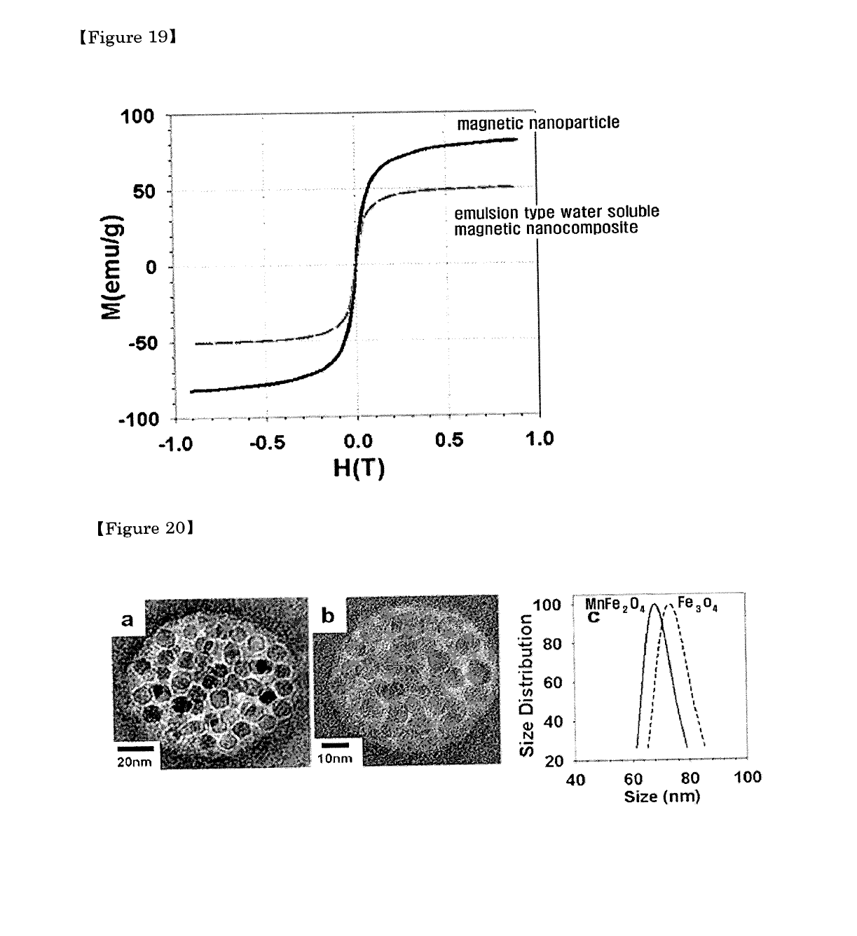

600 mg of a fatty acid amphiphilic polymer, monomethoxypolyethyleneglycol-dodecanoic acid, prepared in Preparation Example 4 above was dissolved in 20 ml of deionized water as an aqueous phase. 20 mg of magnetic nanoparticles prepared in Preparation Example 1 were dissolved in 5 ml of chloroform as an oil phase. The aqueous phase was mixed with the oil phase, and then the mixture was saturated for 10 minutes by ultrasound of 300 W. The resulting emulsion was stirred for 6 h to vaporize the oil phase, and subjected to centrifugation and a gel filtration column (Sephacryl S-300) to prepare the magnetic nanocomposite for high sensitive MRI with removed impurities. The schematic diagram of the emulsion type magnetic nanocomposite using the fatty acid amphiphilic polymer, monomethoxypolyethyleneglycol-dodecanoic acid, was depicted in FIG. 3c. The prepared particles were identified by a transmission electron microscope and a dynamic laser light scattering method, and the results were depicted in FIGS. 18a and 18b, respectively. The magnetic property was identified as superparamagnetism by vibration sample magnetometer, and the result was depicted in FIG. 19. A solid line represents a hysteresis loop of magnetic nanoparticles, and a dotted line represents a hysteresis loop of the emulsion type magnetic nanocomposite using a fatty acid amphiphilic compound. In addition, the presence of an amphiphilic polymer, monomethoxypolyethyleneglycol-dodecanoic acid and magnetic nanoparticles was identified by IR spectroscopy, and the result was depicted in FIG. 7, line (d).

### Example 4

### Preparation of an Emulsion Type Magnetic Nanocomposite that the Binding Part for a Hydrophilic Active Ingredient is Substituted with a Carboxyl Group, Using a Biodegradable Amphiphilic Polymer

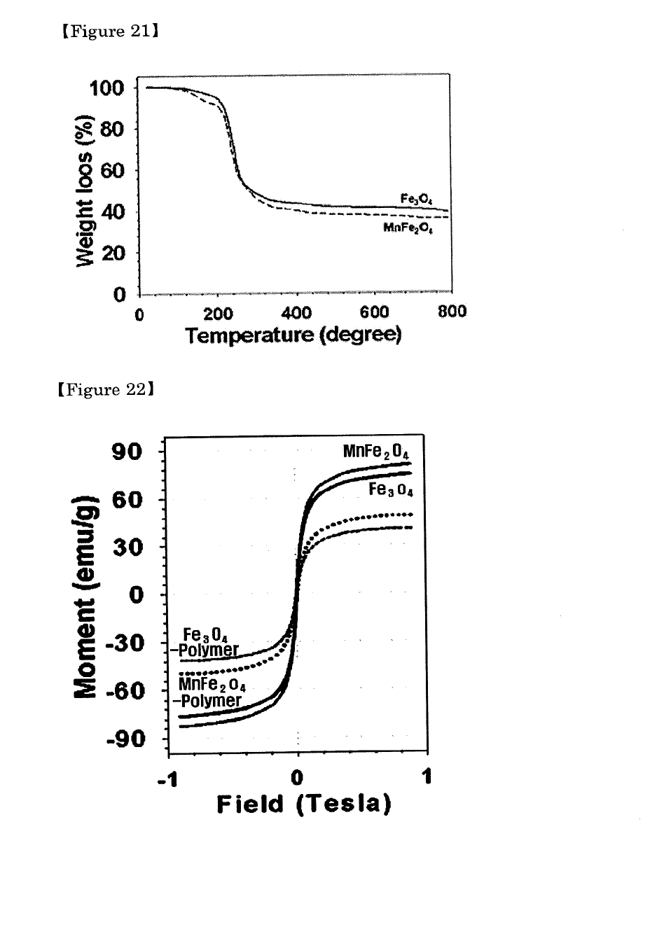

100 mg of Amphiphilic biodegradable polymer prepared in Preparation Example 5, A. above was dissolved in 20 ml of deionized water as an aqueous phase. 20 mg of magnetite and manganese ferrite prepared in Preparation Example I as magnetic nanoparticles were dissolved in 5 ml of chloroform as an oil phase, together with 2 mg of doxorubicin. The aqueous phase was mixed with the oil phase, and then the mixture was saturated for 10 minutes by ultrasound of 300 W. The resulting emulsion was stirred for 12 h to vaporize the oil phase, and subjected to centrifugation and a gel filtration column (Sephacryl S-300) to prepare the magnetic nanocomposite with removed impurities. The schematic diagram of the emulsion type magnetic nanocomposite that said anticancer agent was enclosed and the binding part for a hydrophilic active ingredient was substituted with a carboxyl group, was depicted in FIG. 3d. The prepared particles were identified by a transmission electron microscope and a dynamic laser light scattering method, and the results were depicted in FIG. 20. In FIG. 20, (a) represents a photograph of the emulsion type magnetic nanocomposite in which magnetite (Fe3U4) is enclosed, (b) represents a photograph of the emulsion type magnetic nanocomposite in which manganese ferrite (MnFe₃O₄) is enclosed, and (c) represents a view of size distribution of the magnetic nanocomposite. In addition, the weight ratio of the enclosed magnetic nanoparticles was analyzed by a thermogravimetric analysis method, and the result was depicted in FIG. 21. The magnetic property was identified by VSM, and the result was depicted in FIG. 22.

### Example 5

### Preparation of the Suspension Type Magnetic Nanocomposite that the Binding Part for a Hydrophilic Active Ingredient is Substituted with a Carboxyl Group Using a Biodegradable Amphiphilic Polymer

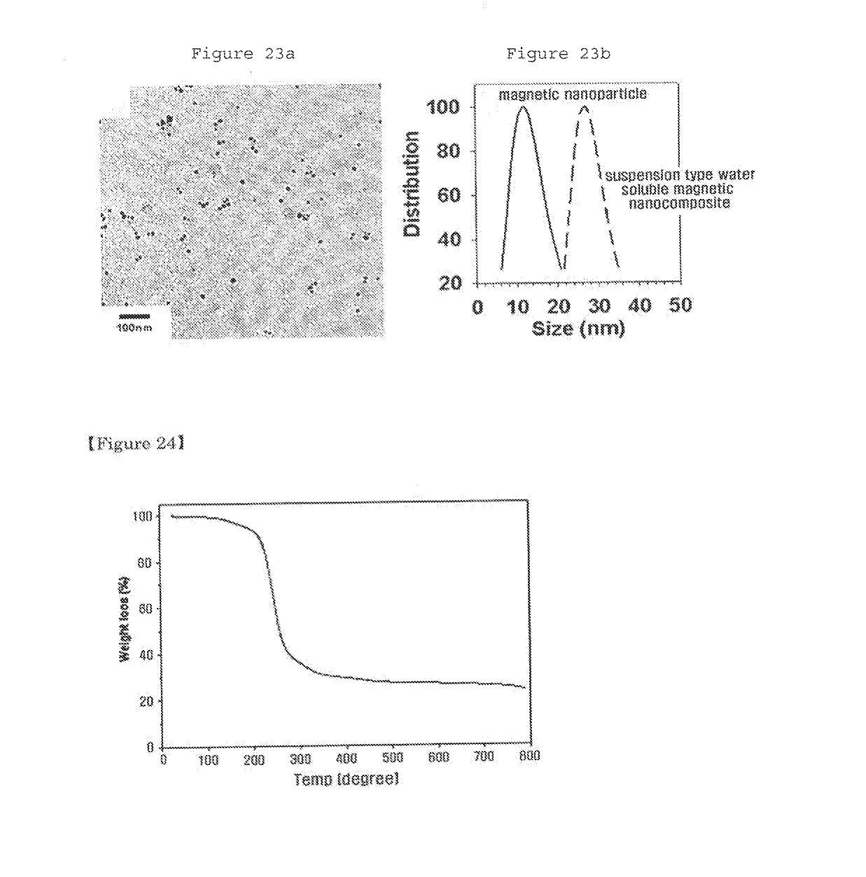

3 mg of Magnetic nanoparticles prepared in Preparation Example 1 above were dispersed in a chloroform solution dissolving 50 mg of the amphiphilic biodegradable polymer prepared in Preparation Example 5, B. above. The dispersion was heated to 40° C., with stirring, to vaporize the solvent, and re-dispersed in 0.5 ml of a phosphate buffered saline (PBS) solution. The solution was heated/stirred at 30° C. for 6 h to complete the suspension. After removing micelles without magnetic particles through centrifugation, the suspension was re-dispersed in 0.5 ml of a PBS solution. The schematic diagram of the suspension type magnetic nanocomposite that the binding part for a hydrophilic active ingredient was substituted with a carboxyl group was depicted in FIG. 3e. The prepared particles were identified by a transmission electron microscope and a dynamic laser light scattering method, and the results were depicted in FIGS. 23a and 23b, respectively. The weight ratio of the enclosed magnetic nanoparticles was analyzed by a thermogravimetric analysis method, and the result was depicted in FIG. 24.

### Example 6

### Preparation of the Emulsion Type Magnetic Nanocomposite that the Binding Part for a Hydrophilic Active Ingredient is Substituted with a Carboxyl Group Using a Commercially Available Surfactant

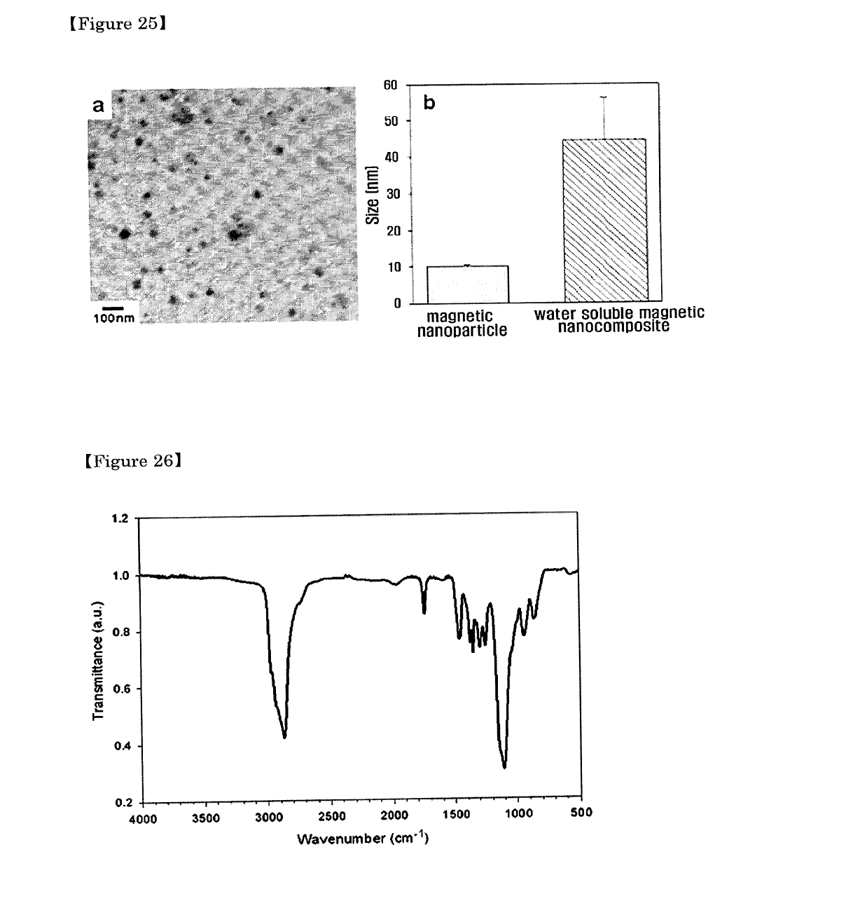

1 g of the amphiphilic polymer prepared in Preparation Example 6 above was dissolved in 40 ml of deionized water as an aqueous phase. 30 mg of magnetic nanoparticles prepared in Preparation Example 1 above were dissolved in 5 ml of hexane as an oil phase. The aqueous phase was mixed with the oil phase, and then the mixture was stirred for 10 minutes with applying ultrasound of 190 W. Then, the mixture was stirred for 30 minutes in the absence of the ultrasound, and saturated for further 10 minutes with applying ultrasound of 600 W. The resulting emulsion was stirred for 24 h to vaporize the oil phase and prepare the magnetic nanocomposite for high sensitive MRI. The schematic diagram of the emulsion type magnetic nanocomposite that the binding part for a hydrophilic active ingredient was substituted with a carboxyl group using a commercially available surfactant was depicted in FIG. 3f. The prepared particles were identified by a transmission electron microscope and a dynamic laser light scattering method, and the results were depicted in picture (a) and graph (b) of FIG. 25. The presence of the amphiphilic polymer Pluronic F-127 and magnetic nanoparticles in the prepared nanocomposite was identified by IR spectroscopy, and the result was depicted in FIG. 26.

### Example 7

### Preparation of Novel Tumor Specific Intelligent Contrast Agent for MRI

Using magnetic nanocomposite prepared in Example 6 above, a tumor specific intelligent contrast agent for MRI was prepared by the following process. Magnetic nanocomposite prepared in Example 6, herceptin as an antibody for treatment [a mole ratio of nanocomposites to herceptin 100:1], and NHS(N-hydroxysuccinimide) and EDC(N-(3-Dimethylaminopropyl)-N-ethyl-carbodiimide hydrochloride) as a cross-linking agent (a mole ratio of NHS to EDC 1:2) were mixed with 2 ml of a PBS buffer, and the mixture was reacted for about 6 h. After completing the reaction, impurities were removed using gel filtration column.

### Example 8

### Preparation of the Emulsion Type Magnetic Nanocomposite that the Binding Part for a Hydrophilic Active Ingredient is Substituted with a Succinimidyl Group

To prepare an emulsion type water soluble magnetic nanocomposite that an anticancer agent was enclosed and the binding part for a hydrophilic active ingredient was substituted with a succinimidyl group (FIG. 3g), chloroform was used as an oil phase, in which 2 mg of doxorubicin (DOX) was dissolved and 20 mg of magnetic nanoparticles prepared in Preparation Example 1 were dispersed. 20 ml of deionized water was used as an aqueous phase, in which 100 mg of the amphiphilic biodegradable polymer prepared in Preparation Example 7 was dissolved. After both phases were mixed to be saturated, the mixture was emulsified for 10 minutes by ultrasound. This emulsion was stirred for 12 h to vaporize the oil phase, and subjected several times to centrifugation and Sephacryl S-300 column to obtain the high pure water soluble magnetic nanocomposite.

### Example 9

### Preparation of the Suspension Type Magnetic Nanocomposite that the Binding Part for a Hydrophilic Active Ingredient is Substituted with a Carboxyl Group

A. Preparation of the Suspension Type Magnetic Nanocomposite in which an Anticancer Agent is Only Physically Enclosed

To prepare the suspension type water soluble magnetic nanocomposite that an anticancer agent was only physically enclosed and the binding part for a hydrophilic active ingredient was substituted with a carboxyl group (FIG. 3h), 3 mg of magnetic nanoparticles prepared in Preparation Example 1 and 2 mg of DOX were dispersed in chloroform in which 50 mg of the amphiphilic biodegradable polymer prepared in Preparation Example 5, B. was dissolved. The dispersion was heated to 40° C., with stirring, to vaporize the solvent, and re-dispersed in 0.5 ml of a PBS solution. The solution was heated/stirred at 30° C. for 6 h to complete the suspension. After removing micelles without magnetic nanoparticles through centrifugation, the suspension was re-dispersed in 0.5 ml of a PBS solution.

B. Preparation of the Suspension Type Magnetic Nanocomposite that an Anticancer Agent is Only Chemically Enclosed

To prepare the suspension type water soluble magnetic nanocomposite that an anticancer agent was only chemically enclosed and the binding part for a hydrophilic active ingredient was substituted with a carboxyl group (FIG. 3h), 3 mg of magnetic nanoparticles prepared in Preparation Example 1 was dispersed in chloroform in which 50 mg of the amphiphilic biodegradable polymer binding the anticancer agent prepared in Preparation Example 8 was dissolved. The dispersion was heated to 40° C., with stirring, to vaporize the solvent, and re-dispersed in 0.5 ml of a PBS solution. The solution was heated/stirred at 30° C. for 6 h to complete the suspension. After removing micelles without magnetic nanoparticles through centrifugation, the suspension was re-dispersed in 0.5 ml of a PBS solution.

C. Preparation of the Suspension Type Magnetic Nanocomposite that an Anticancer Agent is Enclosed by the Physical Method and the Chemical Method

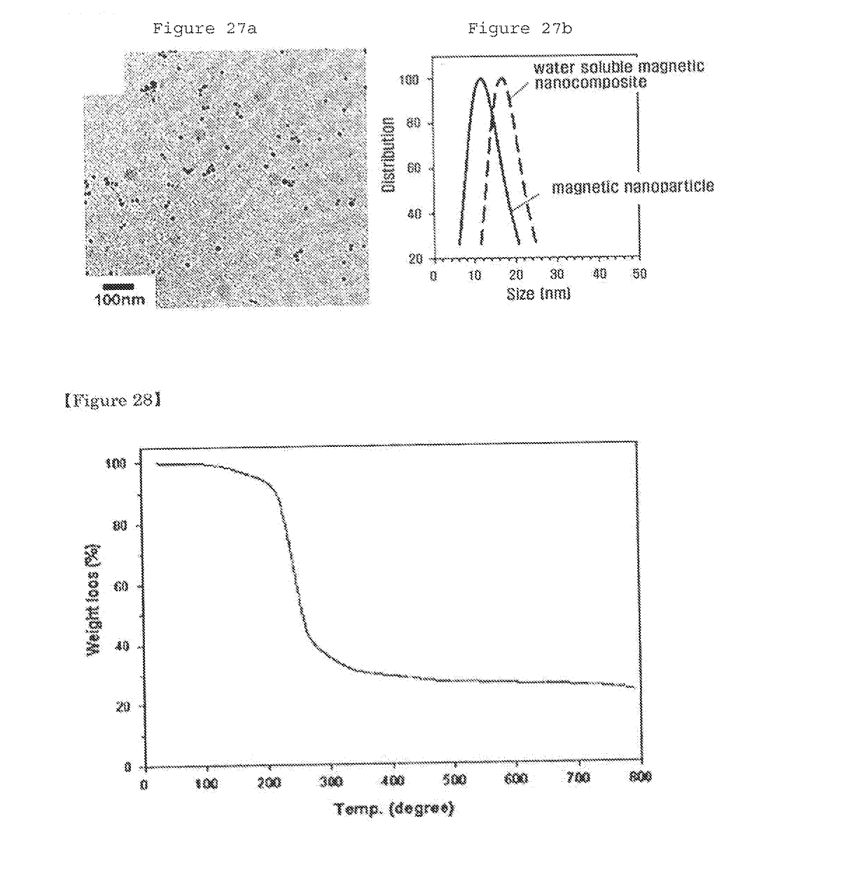

To prepare the suspension type water soluble magnetic nanocomposite that an anticancer agent was enclosed by the physical method and the chemical method and the binding part for a hydrophilic active ingredient was substituted with a carboxyl group (FIG. 3h), 3 mg of magnetic nanoparticles prepared in Preparation Example 1 and 2 mg of DOX were dispersed in chloroform in which 25 mg of the amphiphilic biodegradable polymer prepared in Preparation Example 5, B. and 25 mg of the amphiphilic biodegradable polymer binding the anticancer agent prepared in Preparation Example 8 were dissolved. The dispersion was heated to 40° C., with stirring, to vaporize the solvent, and re-dispersed in 0.5 ml of a PBS solution. The solution was heated/stirred at 30° C. for 6 h to complete the suspension. After removing micelles without magnetic nanoparticles through centrifugation, the suspension was re-dispersed in 0.5 ml of a PBS solution. The prepared particles were identified by a transmission electron microscope and a dynamic laser light scattering method, and the results were depicted in FIG. 27a and 27b, respectively. The weight ratio of the enclosed magnetic nanoparticles was analyzed by a thermogravimetric analysis method, and the result was depicted in FIG. 28.

### Example 10

### Preparation of Herceptin-Mgenetic Nanocomposite for Simultaneous Diagnosis and Treatment of Cancer

A. Preparation of Herceptin-Mgenetic Nanocomposite Using Succinimidyl Group-Magnetic Nanocomposite

The reaction of the herceptin-magnetic nanocomposite was subjected at room temperature for 4 h, by dispersing 3 mg of the water soluble magnetic nanocomposite prepared in Example 8 above in a PBS solution of pH 7.4 and adding 0.1 mg of herceptin thereto. After completing the reaction, the unreacted herceptin and water soluble magnetic nanocomposite was removed via Separcryl S-300 column to prepare the herceptin-magnetic nanocomposite.

B. Preparation of Herceptin-Magnetic Nanocomposite Using the Carboxyl Group-Magnetic Nanocomposite

The magnetic nanocomposite that the terminal functional group of hydrophilic polymer was substituted with a carboxyl group, prepared in Example 4 above, were dispersed in 0.5 ml of a PBS solution. The reaction was subjected at room temperature for 4 h, after dispersing the water soluble magnetic nanocomposite in a PBS solution of pH 7.4 and adding 0.5 mg of herceptin thereto. After completing the reaction, the unreacted herceptin and water soluble magnetic nanocomposite was removed via Separcryl S-300 column to prepare the herceptin-magnetic nanocomposite. To identify cell selectivity of the magnetic nanocomposite bound to an antibody, immunoglobulin (IgG) which does not react with a target cell was bound to magnetic nanocomposite by the method above to prepare IgG-magnetic nanocomposite.

C. Preparation of Herceptin-Magnetic Nanocomposite Using the Carboxyl Group-Magnetic Nanocomposite

The magnetic nanocomposite that the terminal functional group of hydrophilic polymer was substituted with a carboxyl group, prepared in Example 9, C. above, were dispersed in 0.5 ml of a PBS solution. The reaction was subjected at room temperature for 4 h, after dispersing the water soluble magnetic nanocomposite in a PBS solution of pH 7.4 and adding 0.5 mg of herceptin thereto.

After completing the reaction, the unreacted herceptin and water soluble magnetic nanocomposite was removed via Separcryl S-300 column to prepare the herceptin-magnetic nanocomposite. To identify cell selectivity of the magnetic nanocomposite bound to an antibody, immunoglobulin (IgG) which does not react with a target cell was bound to magnetic nanocomposite by the method above to prepare IgG-magnetic nanocomposite.

### Example 11

### Preparation of the Emulsion Type Magnetic Nanocomposite that the Binding Part for a Hydrophilic Active Ingredient is Substituted with a Carboxyl Group

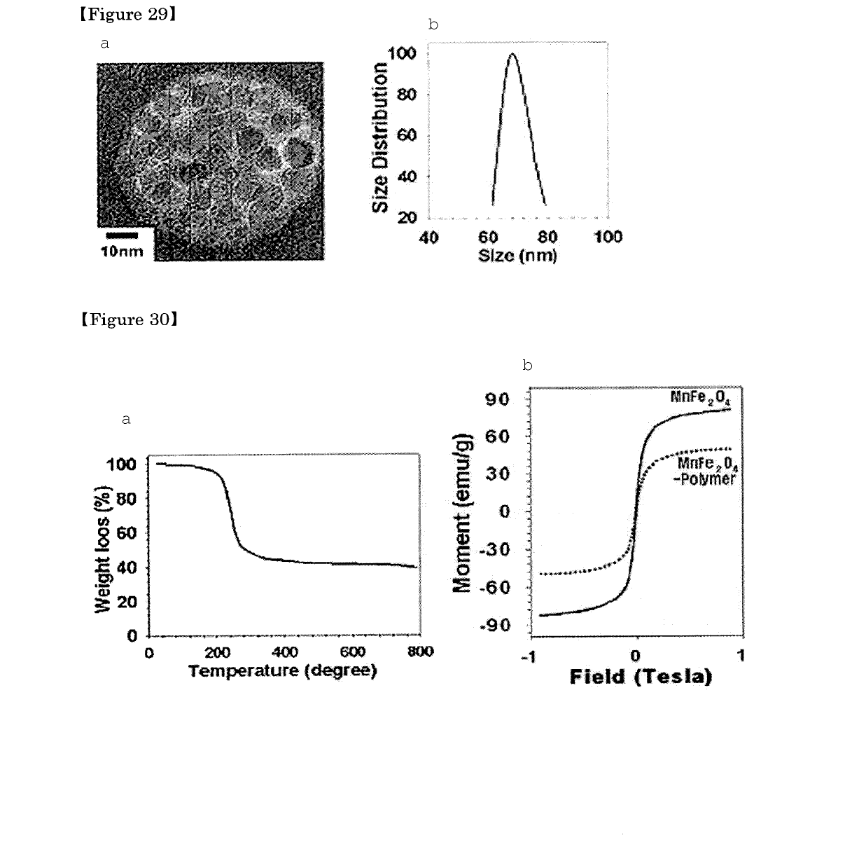

To prepare an emulsion type water soluble magnetic nanocomposite that the binding part for a hydrophilic active ingredient was substituted with a carboxyl group, chloroform was used as an oil phase, in which 100 mg of the amphilic biodegradable polymer prepared in Preparation Example 3 above was dissolved and 20 mg of magnetic nanoparticles prepared in Preparation Example 1 was dispersed. To give fluorescence to the magnetic nanocomposite, 2 mg of Nile red was added to the oil phase. 20 ml of deionized water was used as an aqueous phase. After both phases were mixed to be saturated, the mixture was emulsified for 10 minutes by ultrasound. This emulsion was stirred for 12 h to vaporize the oil phase, and subjected several times to centrifugation and Sephacryl S-300 column to obtain the high pure water soluble magnetic nanocomposite. The prepared particles were identified by a transmission electron microscope and a dynamic laser light scattering method, and the results were depicted in photograph (a) and graph (b) of FIG. 29. The weight ratio of the enclosed magnetic nanoparticles was analyzed by a thermogravimetric analysis method and the magnetic property was measured by VSM, and the results were depicted in graphs (a) and (b) of FIG. 30.

### Example 12

### Preparation of Herceptin-Magnetic Nanocomposite for Separating Cells by Magnetic Field

The magnetic nanocomposite that the terminal functional group of hydrophilic polymer was substituted with a carboxyl group, prepared in Example 11, C. above, were dispersed in 0.5 ml of a PBS solution. The reaction was subjected at room temperature for 4 h, after dispersing the water soluble magnetic nanocomposite in a PBS solution of pH 7.4 and adding 0.5 mg of herceptin thereto. After completing the reaction, the unreacted herceptin and water soluble magnetic nanocomposite was removed via Separcryl S-300 column to prepare the herceptin-magnetic nanocomposite. It was identified in pictures a and b of FIG. 31 to sensitively arrange the magnetic nanocomposite bound to the antibody in an external magnetic field (Nb—B—Fe magnet, 0.35 T). To identify cell selectivity of the magnetic nanocomposite bound to an antibody, immunoglobulin (IgG) which does not react with a target cell was bound to magnetic nanocomposite by the method above to prepare IgG-magnetic nanocomposite.

### Experimental Example 1

### Experiment of Stability in the Emulsion Type Magnetic Nanocomposite Using Biodegradable Aniphiphilic Polymer

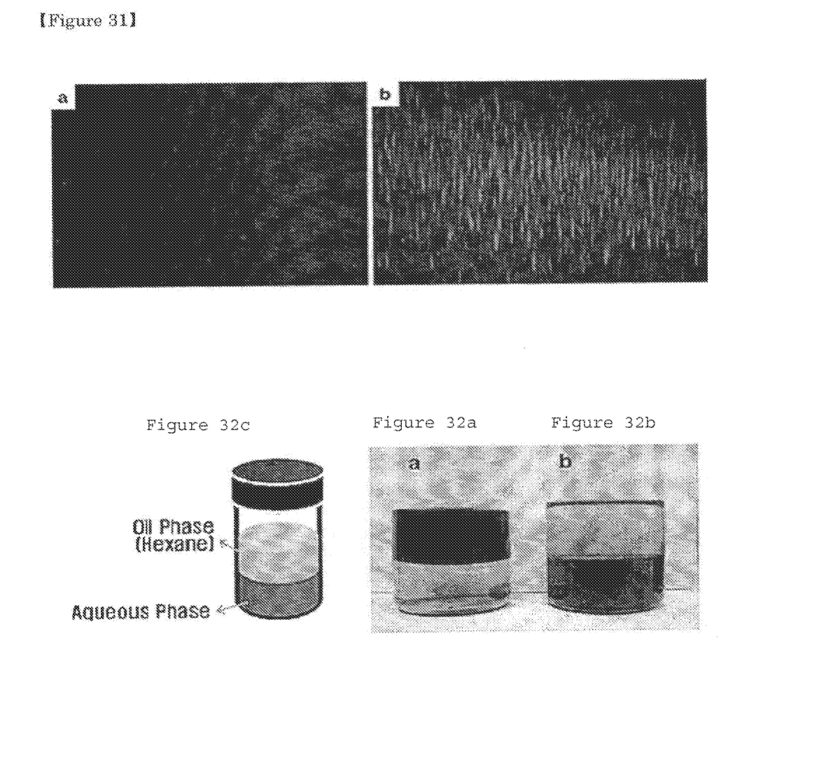

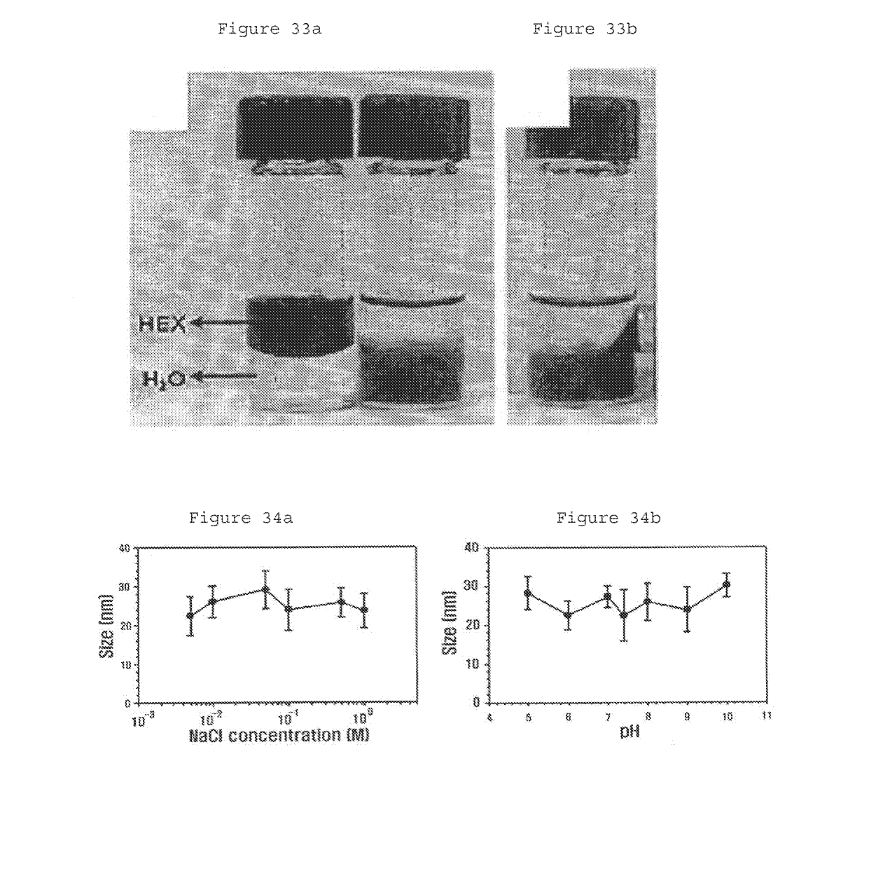

The organic magnetic nanoparticles in prepared in Preparation Example 1 was dissolved in hexane, and water was added thereto. In addition, the emulsion type magnetic nanocomposite using the biodegradable amphiphilic polymer prepared in Example 1 was dissolved in water, and hexane was added thereto. Analyzing the change of solubility, the results were depicted in FIGS. 32a-c. As shown in FIGS. 32a-c, it could be identified that the organic nanoparticle (FIG. 32a) having a fatty acid surface stabilizer to its surface was transformed into a water soluble nanocomposite (FIG. 32b). Upon viewing by naked eye, precipitation or aggregation was not caused. Thus, it could be known that the water soluble iron oxide nanoparticle is well dispersed in an aqueous solution.

### Experimental Example 2

### Experiment of Stability in the Suspension Type Magnetic Nanocomposite Using a Fatty Acid Amphiphilic Polymer

The organic magnetic nanoparticles in prepared in Preparation Example 1 were dissolved in hexane, and water was added thereto. In addition, the emulsion type magnetic nanocomposite using the biodegradable amphiphilic polymer prepared in Example 3 was dissolved in water, and hexane was added thereto. Analyzing the change of solubility, the results were depicted in FIGS. 33a and b. As shown in FIGS. 33a and b, it could be identified that the organic nanoparticle (FIG. 33a, left) having a fatty acid surface stabilizer to its surface was transformed into a water soluble nanocomposite (FIG. 33a, right). When an external magnetic field (Nd—B—Fe magnet, 0.35 T) was applied thereto, the sensitive response could be identified (FIG. 33b). In addition, upon viewing by naked eye, precipitation or aggregation was not caused. Thus, it could be known that the water soluble iron oxide nanoparticle is well dispersed in an aqueous solution.

Stability of the nanocomposite prepared in Example 3 was examined according to concentrations of a salt (NaCl) and pH, and the results were depicted in FIGS. 34a and b. It could be identified from FIG. 34a, a graph representing the size change of nanocomposite according to a concentration of 0.0˜1.0 M, that the size of nanocomposite according to concentration was not nearly changed. It could be also identified from FIG. 34b, a graph representing the size change of nanocomposite according to pH 5˜pH 10, that the size of nanocomposite according to pH was not nearly changed.

### Experimental Example 3

### Experiment of Stability in the Emulsion Type Magnetic Nanocomposite that the Binding Part for a Hydrophilic Active Ingredient is Substituted with a Carboxyl Group, Using a Commercially Available Surfactant

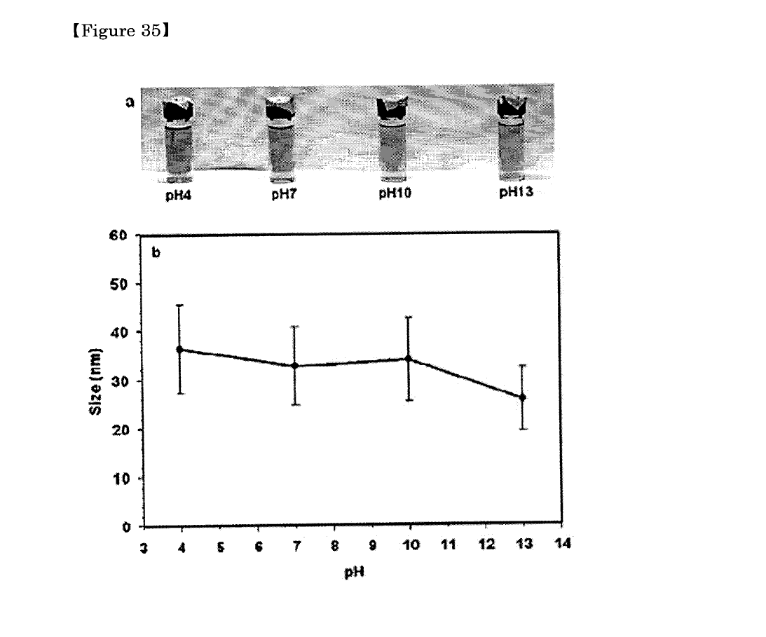

The results of examining dispersion stability of nanocomposite prepared in Example 6 according to pH were depicted in picture (a) and graph (b) of FIG. 35. As shown in FIG. 35, the particle aggregation of nanocomposite could not be identified in a range of pH 4˜13, and the size change of particles could be hardly identified. In addition, stability was examined according to concentrations of salt (NaCl), and the results were depicted in picture (a) and graph (b) of FIG. 36. As shown in FIG. 36, the particle aggregation of nanocomposite could not be identified in a concentration of from 0.005 M to 1.0 M, and the size change of particles could be hardly identified.

### Experimental Example 4

### Identification of Possibility of the Emulsion Type Magnetic Nanocomposite Using the Biodegradable Amphiphilic Polymer as a Contrast Agent

To identify the contrasting effect for MRI of the water soluble magnetic nanocomposite, the water soluble magnetic nanocomposite prepared in Example 1 above was titrated in 0.1, 0.05, 0.025 and 0.125 μg/μl and injected into PCR tubes. 1.5 T system (Intera; Philips Medical Systems, Best, The Netherlands) was used for the contrasting effect of MRI, employing micro-47 coil. Coronal images were obtained with Fast Field Echo (FFE) pulse sequence. Specific parameters were as follows: resolution 156 156 μm, slice thickness 0.6 mm, TE=20 ms, TR=400 ms, number of image excitation 1, time of image acquisition 6 minutes.

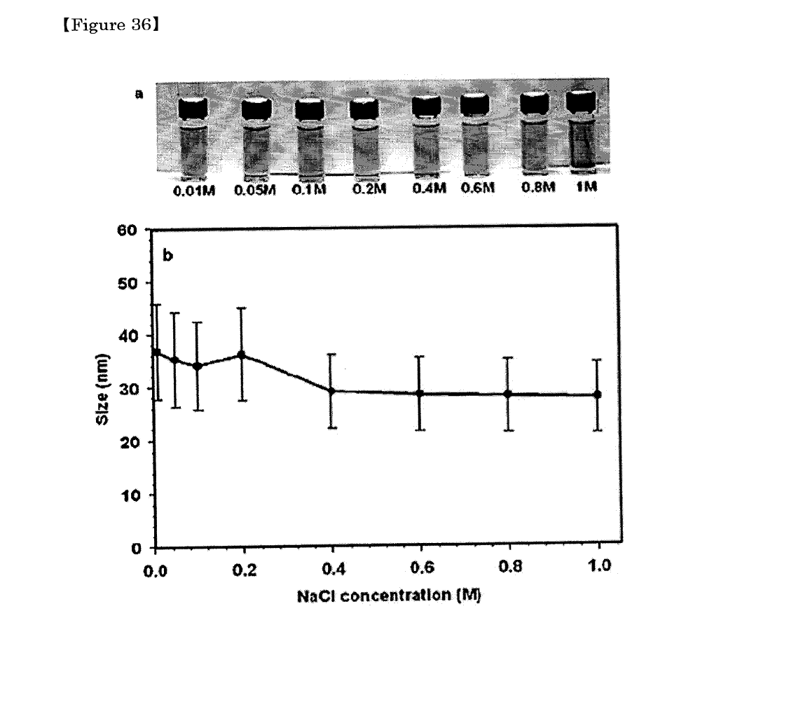

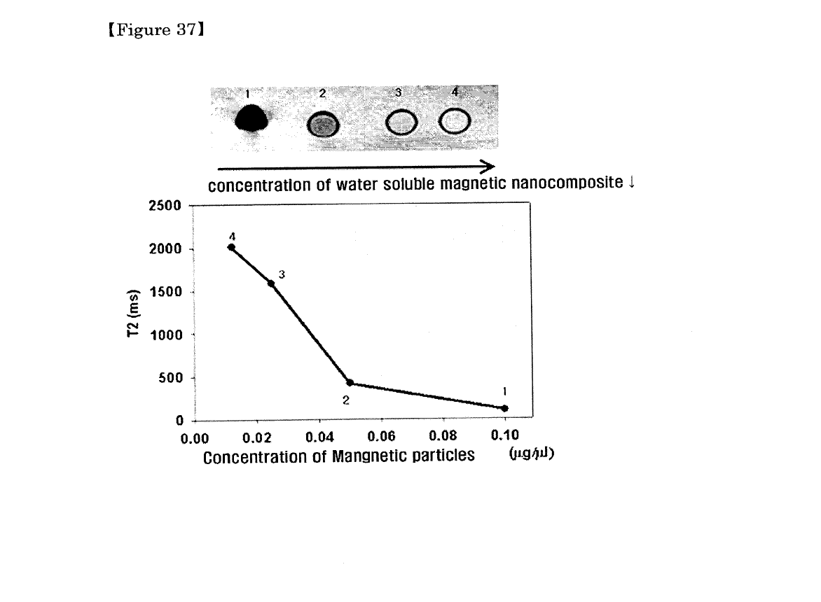

As shown in of FIG. 37, it could be identified that the higher the concentration of water soluble magnetic nanocomposite was, the more the signals of MRI were amplified.

### Experimental Example 5

### Identification of Possibility of the Emulsion Type Magnetic Nanocomposite Using the Fatty Acid Amphiphilic Polymer as a Contrast Agent

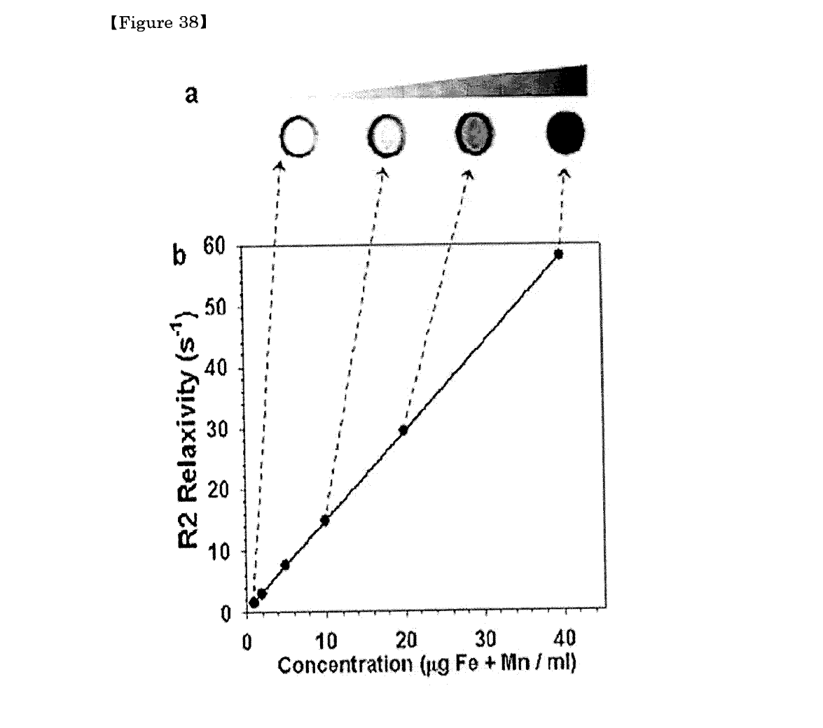

To identify the contrasting effect for MRI of water soluble magnetic nanocomposite, the water soluble magnetic nanocomposite prepared in Example 3 above was titrated and injected into micro-tubes. 1.5 T system (Intera; Philips Medical Systems, Best, The Netherlands) was used for the contrasting effect of MRI, employing micro-47 coil. Coronal images were obtained with Fast Field Echo (FFE) pulse sequence. Specific parameters were as follows: resolution 156 156 μm, slice thickness 0.6 mm, TE=20 ms, TR=400 ms, number of image excitation 1, time of image acquisition 6 minutes. As shown in picture (a) and graph (b) of FIG. 38, it could be identified that the higher the concentration of water soluble magnetic nanocomposite was, the more the signals of MRI were amplified.

### Experimental Example 6

### Identification of Possibility of the Emulsion Type Magnetic Nanocomposite that the Binding Part for a Hydrophilic Active Ingredient is Substituted with a Carboxyl Group as a Contrast Agent

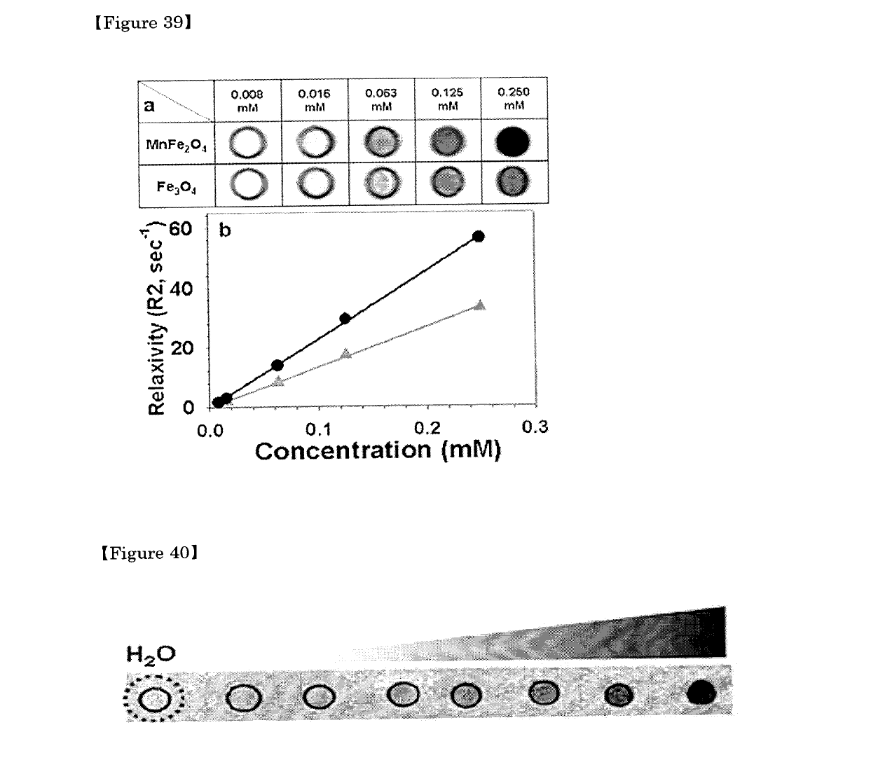

To identify the contrasting effect for MRI of the emulsion type water soluble magnetic nanocomposite that the binding part for a hydrophilic active ingredient was substituted with a carboxyl group, the water soluble magnetic nanocomposite prepared in Example 4 above was titrated and injected into micro-tubes. 1.5 T system (Intera; Philips Medical Systems, Best, The Netherlands) was used for the contrasting effect of MRI, employing micro-47 coil. Coronal images were obtained with Fast Field Echo (FFE) pulse sequence. Specific parameters were as follows: resolution 156 156 slice thickness 0.6 mm, TE=20 ms, TR=400 ms, number of image excitation 1, time of image acquisition 6 minutes. As shown in chart (a) and graph (b) of FIG. 39, it could be identified that the higher the concentration of water soluble magnetic nanocomposite was, the more the signals of MRI were amplified.

### Experimental Example 7

### Identification of Possibility of the Emulsion Type Magnetic Nanocomposite that the Binding Part for a Hydrophilic Active Ingredient is Substituted with a Carboxyl Group, Using a Commercially Available Surfactant, as a Contrast Agent

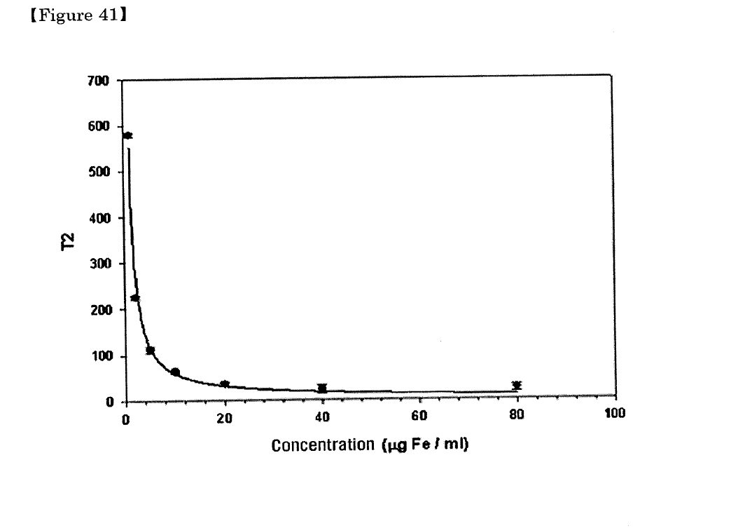

To identify whether the emulsion type magnetic nanocomposite that the binding part for a hydrophilic active ingredient was substituted with a carboxyl group using a commercially available surfactant, prepared in Example 6 shows the sufficient contrasting effect for MRI, the water soluble magnetic nanocomposite was titrated in a concentration of 1.0, 2.0, 5.0, 10.0, 20.0, 40.0 and 80.0 μm/ml and injected into micro-tubes. 1.5 T system (Intera; Philips Medical Systems, Best, The Netherlands) was used for the contrasting effect of MRI, employing micro-47 coil. Coronal images were obtained with Fast Field Echo (FFE) pulse sequence. Specific parameters were as follows: resolution 156 156 μm, slice thickness 0.6 mm, TE=20 ms, TR=400 ms, number of image excitation 1, time of image acquisition 6 minutes. T2 maps were performed to quantitatively evaluate the contrasting effect for MRI. Specific parameters were as follows: resolution 156 156 μm, slice thickness 0.6 mm, TR=4000 ms, TE=20, 40, 60, 80, 100, 120, 140, 160 ms, number of image excitation 2, time of image acquisition 4 minutes. As shown in FIGS. 40 and 41, it could be identified that the higher the concentration of water soluble magnetic nanocomposite was, the more the signals of MRI were amplified. It is indicated that the water soluble magnetic nanocomposite may be used as a nano-contrast agent.

### Experimental Example 8

### Identification of Possibility of the Magnetic Nanocomposite as a Contrast Agent

A. Identification of Possibility of the Emulsion Type Magnetic Nanocomposite that the Binding Part for a Hydrophilic Active Ingredient is Substituted with a Succinimidyl Group as a Contrast Agent

To identify the contrasting effect for MRI of the emulsion type water soluble magnetic nanocomposite that the binding part for a hydrophilic active ingredient was substituted with a succinimidyl group, the water soluble magnetic nanocomposite prepared in Example 8 above was titrated and injected into micro-tubes. 1.5 T system (Intera; Philips Medical Systems, Best, The Netherlands) was used for the contrasting effect of MRI, employing micro-47 coil. Coronal images were obtained with Fast Field Echo (FFE) pulse sequence. Specific parameters were as follows: resolution 156 156 μm, slice thickness 0.6 μm, TE=20 ms, TR=400 ms, number of image excitation 1, time of image acquisition 6 minutes. T2 maps were performed to quantitatively evaluate the MRI contrasting effect for antigen specificity. Specific parameters were as follows: resolution 156 156 μm, slice thickness 0.6 mm, TR=4000 ms, TE=20, 40, 60, 80, 100, 120, 140, 160 ms, number of image excitation 2, time of image acquisition 4 minutes. As shown in pictures (a)-(d) and graph of FIG. 42, it could be identified that the higher the concentration of water soluble magnetic nanocomposite was, the more the signals of MRI were amplified.

B. Identification of Possibility of the Suspension Type Magnetic Nanocomposite that the Binding Part for a Hydrophilic Active Ingredient is Substituted with a Carboxyl Group as a Contrast Agent

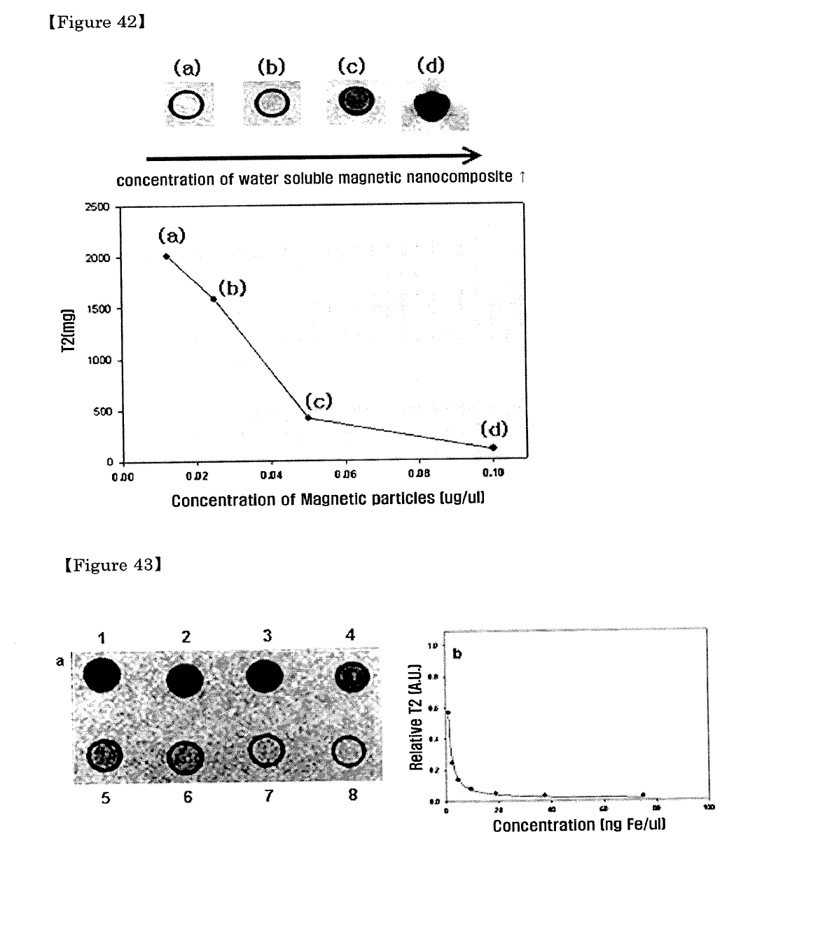

To identify the contrasting effect for MRI of the suspension type water soluble magnetic nanocomposite that the binding part for a hydrophilic active ingredient was substituted with a carboxyl group, the water soluble magnetic nanocomposite prepared in Example 9, C. above was titrated and injected into micro-tubes. 1.5 T system (Intera; Philips Medical Systems, Best, The Netherlands) was used for the contrasting effect of MRI, employing micro-47 coil. Coronal images were obtained with Fast Field Echo (FEE) pulse sequence. Specific parameters were as follows: resolution 156 156 μm, slice thickness 0.6 mm, TE=20 ms, TR=400 ms, number of image excitation 1, time of image acquisition 6 minutes. T2 maps were performed to quantitatively evaluate the MRI contrasting effect for antigen specificity. Specific parameters were as follows: resolution 156 156 μm, slice thickness 0.6 mm, TR=4000 ms, TE=20, 40, 60, 80, 100, 120, 140, 160 ms, number of image excitation 2, time of image acquisition 4 minutes. As shown in picture (a) and graph (b) of FIG. 43, it could be identified that the higher the concentration of water soluble magnetic nanocomposite was, the more the signals of MRI were amplified.

### Experimental Example 9

### Identification of the Binding Degree of Novel Tumor Specific Intelligent Contrast Agent for MRI to Cells and their Contrasting Effect

For novel intelligent contrast agent for MRI with removed impurities prepared in Example 7, the possibility as an intelligent contrast agent was identified through identifying the binding degree of NIH3T6.7 cell positive and MDAMB231 cell negative against an antigen-antibody specific binding to an antibody for treatment, herceptin. Secondary antibody adhered by a fluorescent staining agent (FITC, Fluorescin isothiocyanate) was adhered to the cells, followed by analyzing through FACS, and the result was depicted in FIG. 55. In FIG. 44, (a) represents the fluorescence intensity of the cell unreacted with the intelligent contrast agent for MRI according to the present invention by FACS, (b) represents that of the antibody negative cell (MDAMB231) reacted with the intelligent contrast agent for MRI by FACS, and (c) represents that of the antibody positive cell (NIH3T6.7) reacted with the intelligent contrast agent for MRI by FACS. As shown in FIG. 12, it could be known that the positive NIH3T6.7 cell has higher fluorescence intensity over the negative MDAMB231 cell. It is said from this fact that the prepared contrast agent may be used as the intelligent contrast agent for specifically binding to particular tumor. In addition, the contrasting effect of the NIH3T6.7 cell positive against antibody was identified by MRI, and the result was depicted in FIG. 45.

### Experimental Example 10

### Identification of Cancer Cell Selectivity in the Tumor Specific Magnetic Nanocomposite Via a Flow Cytometry

A. Identification of Cancer Cell Selectivity in the Herceptin-Magnetic Nanocomposite Using the Succinimidyl Group-Magnetic Nanocomposite Via a Flow Cytometry

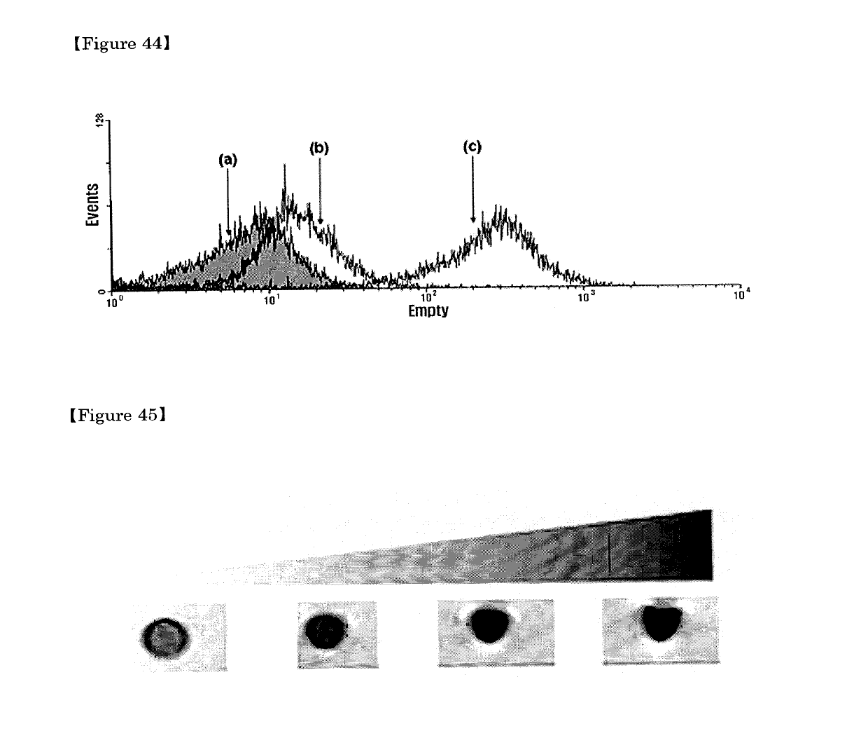

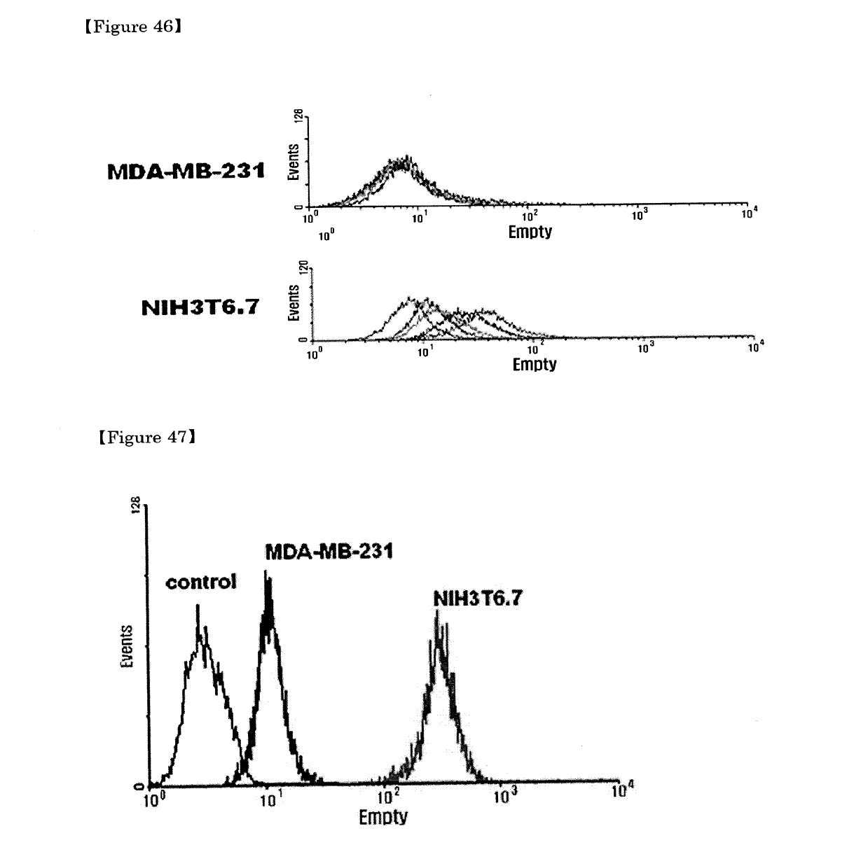

To analyze the binding specificity and efficiency of herceptin-magnetic nanocomposite prepared in Example 10, A. above against a breast cancer labeled antigen, FACS was employed. Each cell line was measured in 10,000 events. Fluorescence intensity distribution in a range of mean value to median value was employed as fluorescence indexes, and the results were depicted in FIG. 46. Herceptin-magnetic nanocomposite and nanocomposite as a control group were treated to cell lines each expressing HER2/new receptor (MDA-MB-231 cell line [symbol] NIH3T6.7 cell line), and reacted with the secondary antibody polymerized with FITC as described above. It could be identified that the intensity of fluorescence expression is increased as the degree of expressing HER2/neu receptor is increased. In case of MDA-MB-231 cell line with a low expression of HER2/neu receptor, it could be shown that the intensity of fluorescence expression is slightly increased relative to the case employing nanocomposite as a control group, and that the intensity of fluorescence expression is gradually increased as the degree of expressing the receptor is increased.

B. Identification of Cancer Cell Selectivity in the Herceptin-Magnetic Nanocomposite Using the Emulsion Type Carboxyl Group-Magnetic Nanocomposite Via a Flow Cytometry

To analyze the binding specificity and efficiency of herceptin-magnetic nanocomposite prepared in Example 10, B. above against a breast cancer labeled antigen, FACS was employed. Each cell line was measured in 10,000 events. Fluorescence intensity distribution in a range of mean value to median value was employed as fluorescence indexes. Herceptin-magnetic nanocomposite and nanocomposite as a control group were treated to cell lines each expressing HER2/new receptor (MDA-MB-231 cell line, NIH3T6.7 cell line), and reacted with the secondary antibody polymerized with FITC as described above. Fluorescence expression was identified using FACS, and the results were depicted in FIG. 47. It could be identified that the intensity of fluorescence expression is increased as the degree of expressing HER2/neu receptor is increased.

C. Identification of Cancer Cell Selectivity in the Herceptin-Magnetic Nanocomposite Using the Suspension Type Carboxyl Group-Magnetic Nanocomposite Via a Flow Cytometry

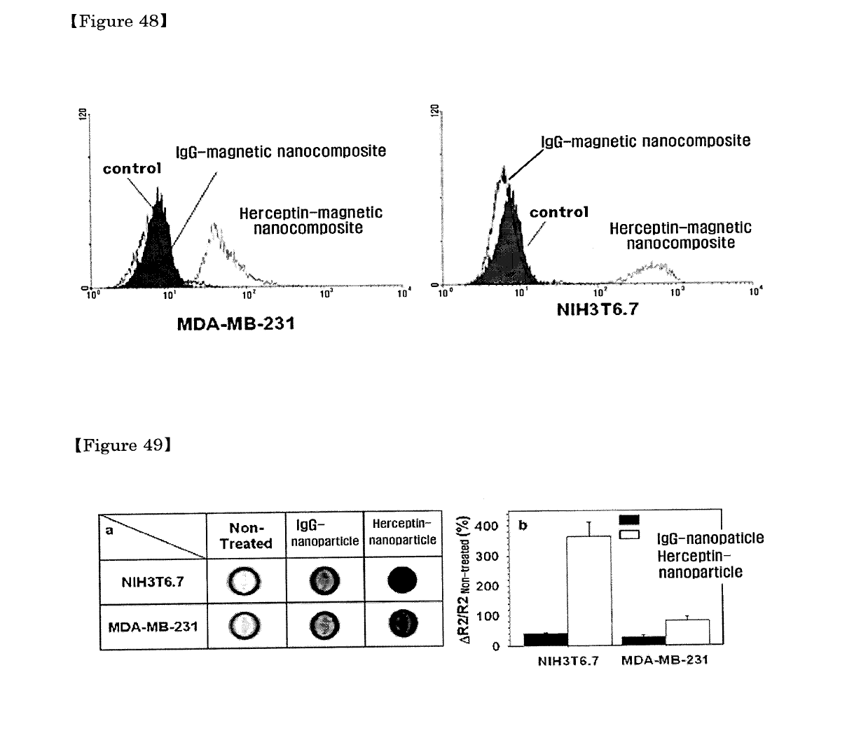

To analyze the binding specificity and efficiency of the herceptin-magnetic nanocomposite prepared in Example 10, C. above against a breast cancer labeled antigen, FACS was employed. Each cell line was measured in 10,000 events. Fluorescence intensity distribution in a range of mean value to median value was employed as fluorescence indexes. Herceptin-magnetic nanocomposite and nanocomposite as a control group were treated to cell lines each expressing HER2/new receptor (MDA-MB-231 cell line, NIH3T6.7 cell line), and reacted with the secondary antibody polymerized with FITC as described above. Fluorescence expression was identified using FACS, and the results were depicted in FIG. 48. It could be identified that the intensity of fluorescence expression is increased as the degree of expressing HER2/neu receptor is increased.

### Experimental Example 11

### Identification of Cell Selectivity in the Tumor Specific Magnetic Nanocomposite Via MRI

A. Identification of Cancer Cell Selectivity in the Emulsion Type Carboxyl Group-Magnetic Nanocomposite Using Herceptin-Magnetic Nanocomposite Via MRI

To analyze antigen specificity of herceptin-magnetic nanocomposite prepared in Example 10, B. above via MRI, each cell was transformed into PCR tubes and then precipitated by centrifugation. 1.5 T system (Intera; Philips Medical Systems, Best, The Netherlands) was used for the contrasting effect of MRI according to antigen specificity of each cell line, employing micro-47 coil. Coronal images were obtained with Fast Field Echo (FFE) pulse sequence, and depicted in picture (a) and graph (b) of FIG. 49. Specific parameters were as follows: resolution 156 156 μm, slice thickness 0.6 mm, TE=20 ms, TR=400 ms, number of image excitation 1, time of image acquisition 6 minutes. T2 maps were performed to quantitatively evaluate the MRI contrasting effect for antigen specificity. Specific parameters were as follows: resolution 156 156 μm, slice thickness 0.6 mm, TR=4000 ms, TE=20, 40, 60, 80, 100, 120, 140, 160 ms, number of image excitation 2, time of image acquisition 4 minutes.

The results in FIG. 49 met with the results of fluorescence expression as shown in FIG. 47. It was identified that the signals of MRI appeared gradually from gray to black, as the degree of expressing HER2/neu receptor was increasing. In case of the cell line with a low expressing degree, it could be identified that the signal turns a little dark color relative to the case employing nanocomposite as a control group, and that it turns gradually black as the degree of expressing the receptor is increased. That is, herceptin-magnetic nanocomposite was selectively bound to the cell line expressing HER2/neu receptor, whereby the signals of MRI appeared gradually black. It could be consequently identified that herceptin-magnetic nanocomposite of the present invention may be used in diagnosing in vitro breast cancer.

B. Identification of Cancer Cell Selectivity in the Suspension Type Carboxyl Group-Magnetic Nanocomposite Using Herceptin-Magnetic Nanocomposite Via MRI

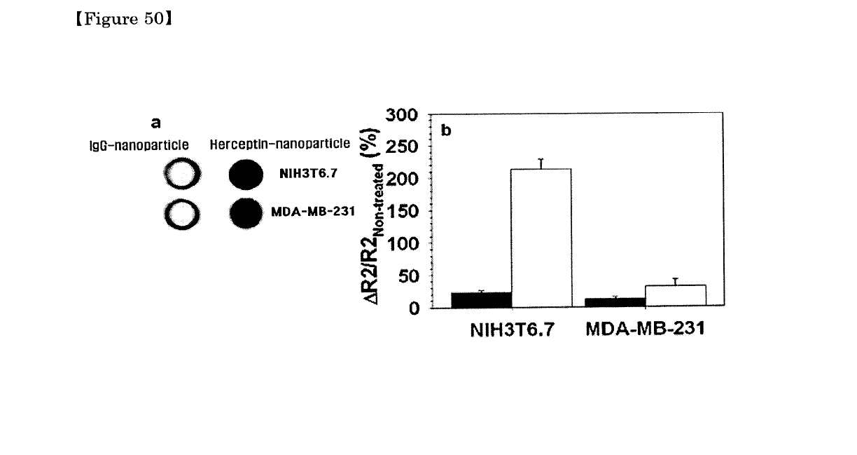

To analyze antigen specificity of herceptin-magnetic nanocomposite prepared in Example 10, C. above via MRI, each cell was transformed into PCR tubes and then precipitated by centrifugation. 1.5 T system (Intera; Philips Medical Systems, Best, The Netherlands) was used for the contrasting effect of MRI according to antigen specificity of each cell line, employing micro-47 coil. Coronal images were obtained with Fast Field Echo (FFE) pulse sequence, and depicted in FIG. 50. Specific parameters were as follows: resolution 156 156 μm, slice thickness 0.6 mm, TE=20 ms, TR=400 ms, number of image excitation 1, time of image acquisition 6 minutes. T2 maps were performed to quantitatively evaluate the MRI contrasting effect for antigen specificity. Specific parameters were as follows: resolution 156 156 μm, slice thickness 0.6 mm, TR=4000 ms, TE=20, 40, 60, 80, 100, 120, 140, 160 ms, number of image excitation 2, time of image acquisition 4 minutes.

The results in drawing (a) and graph (b) of FIG. 50 met with the results of fluorescence expression as shown in FIG. 48. It was identified that the signals of MRI appeared gradually from gray to black, as the degree of expressing HER2/neu receptor was increasing. In case of the cell line with a low expressing degree, it could be identified that the signal turns a little dark color relative to the case employing nanocomposite as a control group, and that it turns gradually black as the degree of expressing the receptor is increased. That is, herceptin-magnetic nanocomposite was selectively bound to the cell line expressing HER2/neu receptor, whereby the signals of MRI appeared gradually black. It could be consequently identified that herceptin-magnetic nanocomposite of the present invention may be used in diagnosing in vitro breast cancer.

### Experimental Example 12

### Analysis of Drug Release Behavior in the Magnetic Nanocomposite

A. Analysis of Drug Release Behavior in the Herceptin-Magnetic Nanocomposite Using the Succinimidyl Group-Magnetic Nanocomposite

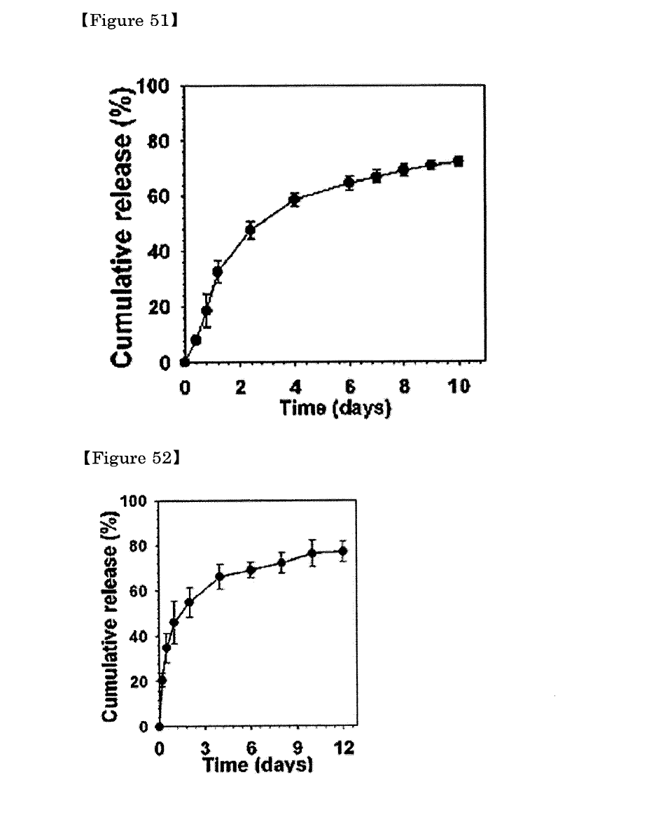

The drug release experiment of water soluble magnetic nanocomposite enclosing anticancer agent prepared in Example 10, A. above was performed by making a titration curve using UV and extracting samples in a certain time interval to measure their concentrations, and the result was depicted in FIG. 51.

B. Analysis of Drug Release Behavior in the Herceptin-Magnetic Nanocomposite Using the Carboxyl Group-Magnetic Nanocomposite

The drug release experiment of water soluble magnetic nanocomposite enclosing anticancer agent prepared in Example 10, B. above was performed by making a titration curve using UV and extracting samples in a certain time interval to measure their concentrations, and the result was depicted in FIG. 52.

C. Analysis of Drug Release Behavior in the Suspension Type Herceptin-Magnetic Nanocomposite

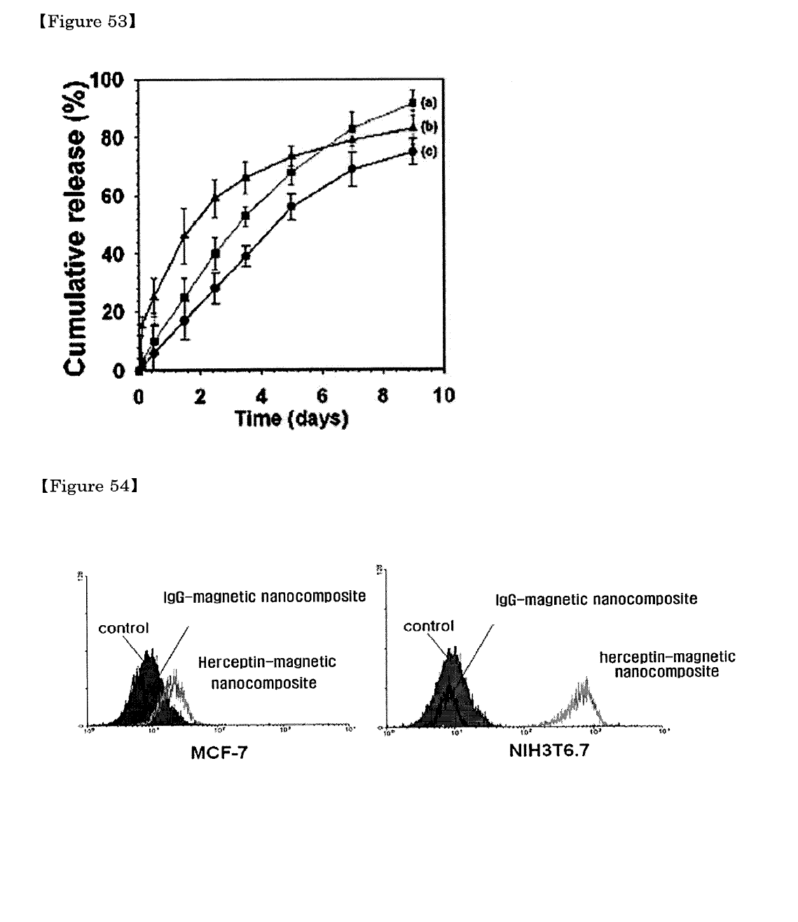

The drug release experiment of water soluble magnetic nanocomposite enclosing anticancer agent prepared in Example 10, C. above was performed by making a titration curve using UV and extracting samples in certain time interval to measure their concentrations, and the results were depicted in FIG. 53. In case of enclosing a drug only by the physical method (FIG. 53, line (b)), the amount of initial release was large. In case of enclosing a drug only by the chemical method (FIG. 53, line (c)), the speed of release was slow, but a linear release behavior was shown. In addition, in case of enclosing the anticancer agent by simultaneously using the physical method and the chemical method, the release mode was linear, and the drug release behavior approaching to 100% for relatively short time was shown (FIG. 53, line (a)).

### Experimental Example 13

### Identification of Target Cell Selectivity in Cell Specific Emulsion Type Herceptin-Magnetic Nanocomposite Via a Flow Cytometry

To analyze the binding specificity and efficiency of herceptin-magnetic nanocomposite prepared in Example 12 above against cancer cell labeled antigen, FACS (Flow cytometer, FACScan, Becton Dickinson, San Diego, Calif.) was employed. Each cell line (MCF-7 cell line [symbol] NIH3T6.7 cell line) was measured in 10,000 events. Fluorescence intensity distribution in a range of mean value to median value was employed as fluorescence indexes. Herceptin-magnetic nanocomposite and a nanocomposite as a control group were treated to cell lines each expressing HER2/new receptor. Then, fluorescence expression was identified using FACS, and the results were depicted in FIG. 54. As shown in FIG. 54, it could be identified that the intensity of fluorescence expression is increased as the degree of expressing HER2/neu receptor is increased. In addition, it could be identified that IgG-magnetic nanocomposite have no cell selectivity.

### Experimental Example 14

To identify possibility of target cell separation using herceptin-magnetic nanocomposite prepared Example 12 above, 1 mg/ml of herceptin-magnetic nanocomposite was incubated in 4*10⁴ NIH3T6.7 for 30 minutes. The unreacted magnetic nanocomposite was separated and inserted in macro-tube. An external magnetic field (Nd—B—Be magnet, 0.35T) was applied on the outside wall of tube. After applying the magnetic field, it was identified using microscope that the nanocomposite was sensitively moved into the direction of magnet within several seconds. The result was depicted in pictures (a) and (b) of FIG. 55.

### Experimental Example 15

### Cytotoxicity Test of the Emulsion Type Magnetic Nanocomposite Using a Fatty Acid Amphphilic Compound as a Contrast Agent

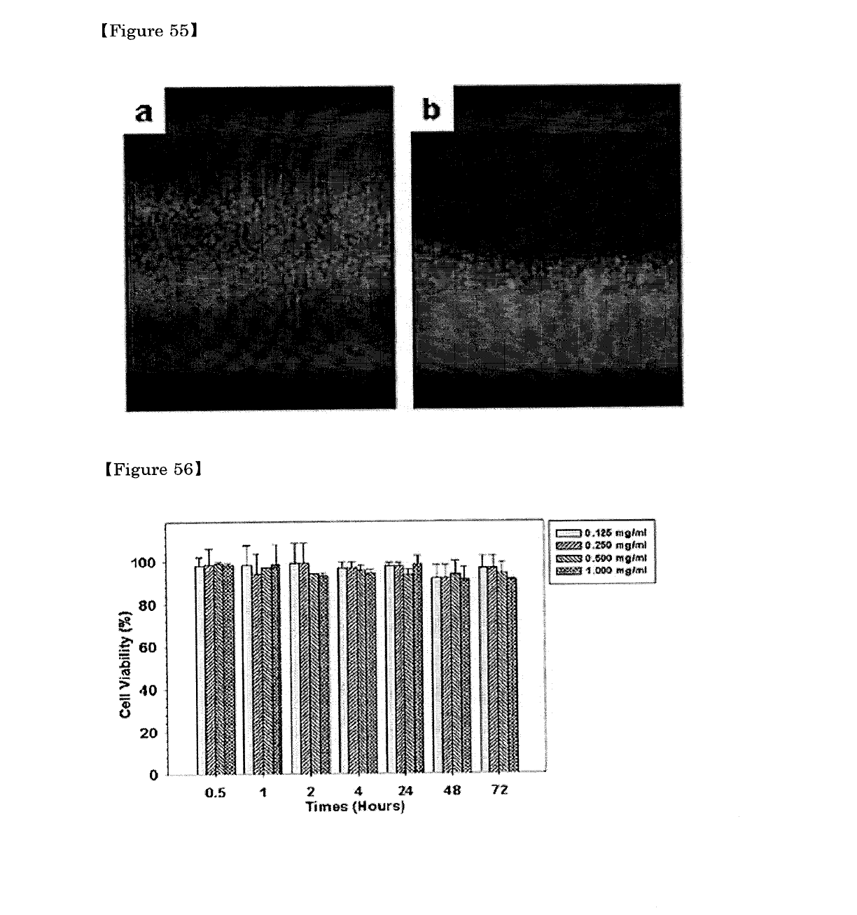

To identify cytotoxicity of water soluble magnetic nanocomposite prepared in Example 3 above, cytotoxicity analysis was proceeded on NIH3T6.7 cell with concentrations of nanocomposite, and the results were depicted in FIGS. 34a and b. The cytotoxicity was identified by examining the concentration of nanocomposite in a range of 10^{-4˜10}⁰ mg/ml and proceeding incubation time of cells for 0˜72 h. As shown in FIG. 56, the cytotoxicity of the magnetic nanocomposite could not be identified at even higher concentrations.

### Experimental Example 16

### Cytotoxicity Test of the Emulsion Type Magnetic Nanocomposite that the Binding Part for a Hydrophilic Active Ingredient was Substituted with a Carboxyl Group Using a Commercially Available Surfactant

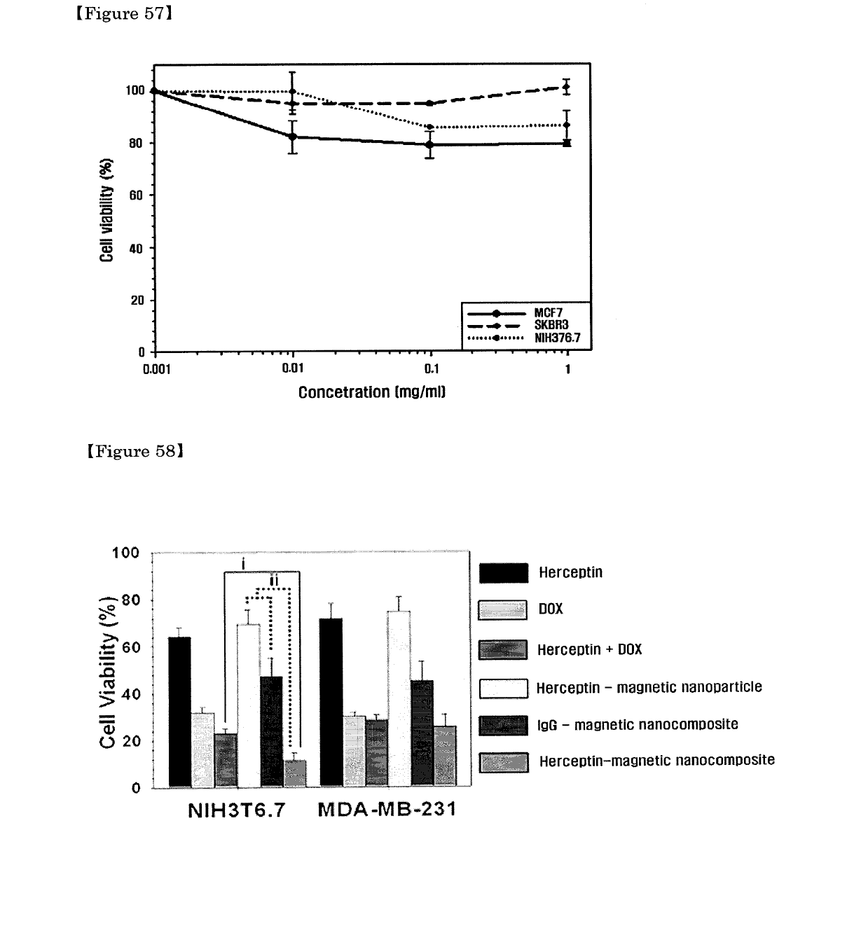

To identify cytotoxicity of the emulsion type magnetic nanocomposite that the binding part for a hydrophilic active ingredient was substituted with a carboxyl group using a commercially available surfactant prepared in Example 6 above, MTT assay was proceeded on MCF7 cell, SKBR3 cell, and NIH3T6.7 cell with concentrations of the nano-contrast agent, and the results were depicted in FIG. 9. As shown in FIG. 57, the cytotoxicity of the magnetic nanocomposite could not be identified at even higher concentrations.

### Experimental Example 17

### Cytotoxicity Test of the Magnetic Nanocomposite

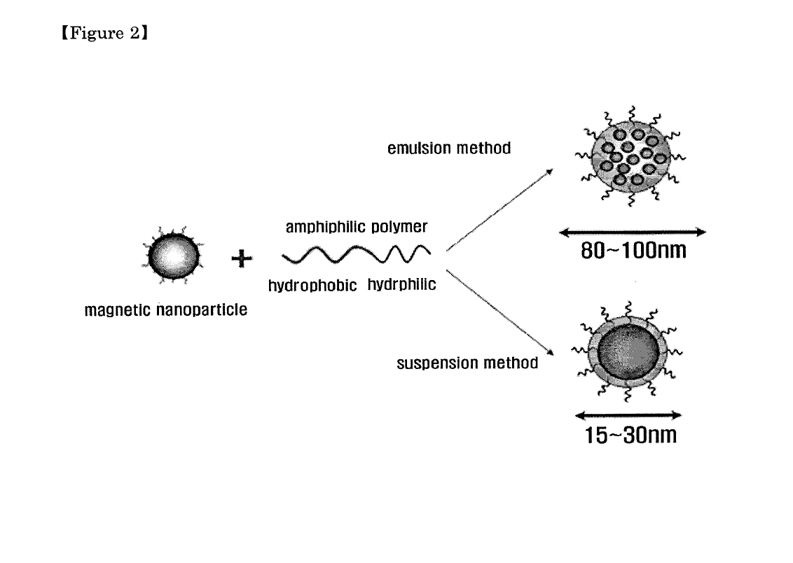

Cytotoxicity test of the prepared magnetic nanocomposite was subjected on NIH3T6.7 cell and MDA-MB-231 cell. The cytotoxicity was identified by representing as a ratio the degree of inhibiting cell growth by DOX alone, herceptin alone, DOX and herceptin, herceptin-magnetic nanoparticles, IgG-magnetic nanocomposite, and herceptin-magnetic nanocomposite. 4*10³ Cells were injected into 96-well, and the test materials were inserted in the cell containing well, based on the equivalent of herceptin and DOX. After 4 h, the residue was washed and the cells were grown for further 72 h. The cytotoxicity obtained from MTT agent was depicted in FIG. 58. The cytotoxicity of herceptin-magnetic nanocomposite enclosing DOX was higher than that of the case acted by herceptin and DOX together (FIG. 26(i)), and much higher than that of the case that herceptin and DOX were reacted with the cells, using nanoparticles. In case of acting herceptin on the cells, NIH3T6.7 cell line showed lower survival rate over MDA-MB-231 cell line. It was also identified that the nanocomposite had cell selectivity. As described in FIG. 2, it can be noted from these results that herceptin-magnetic nanocomposite enclosing DOX is capable to have the synergistic effect of treating cancer cells selectively.

### Experimental Example 18

### Identification of Possibility in the Emulsion Type Magnetic Nanocomposite Using a Fatty Acid Amphiphilic Compound as a Nano-Contrast Agent Via an Animal Model

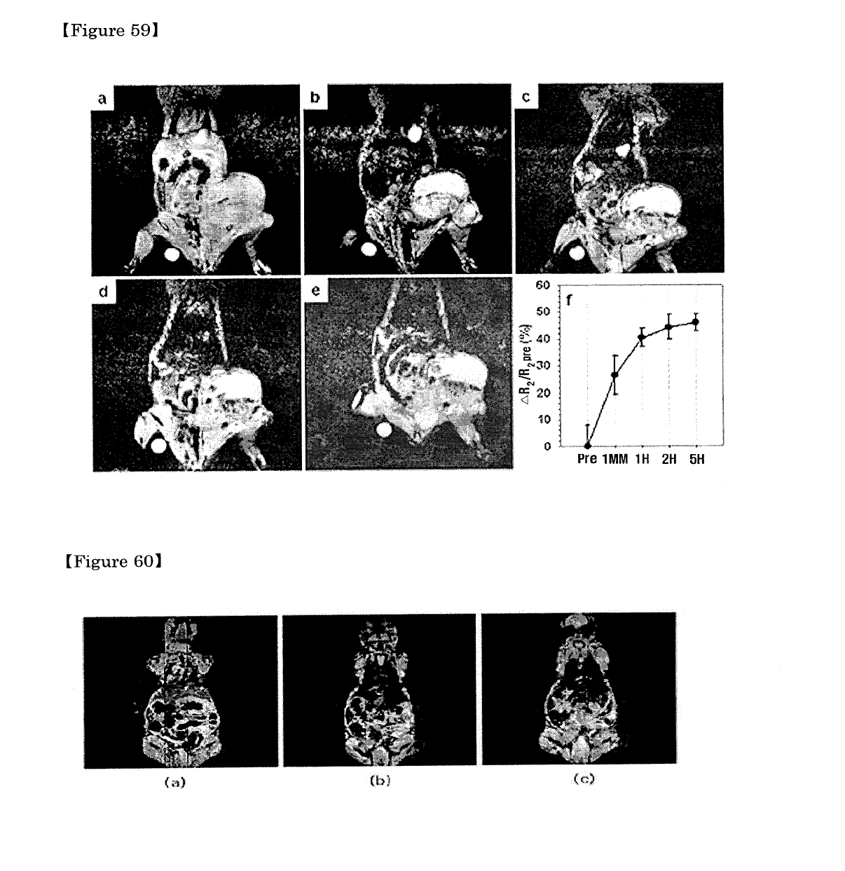

In vivo experiment was subjected, using a nude mouse as an animal model. NIH3T6.7 cells were into the mouse to express cancer cells. After 10 days, the nanocomposite (80 μg Fe+Mn) prepared in Example 3 were injected therein, when the size of cancer cells was 30 mm. MRIs before and after injection were depicted in FIG. 59. That is, there are MRIs before injection FIG. 59, picture (a), just after injection FIG. 59, picture (b), one hour after injection FIG. 59, picture (c), two hours after injection FIG. 59, picture (d), and five hours after injection FIG. 59, picture (e), of the nanocomposite. As represented in FIG. 59, it could be identified that images of liver and cancer cells were apparently changed and the contrasting effect was kept after 1 h, 2 h and 5 h. As a result of making a graph about the change of T2 values over time through the images above, it could be identified that the difference of T2 values after even 5 h is highly kept relative to the value before injection (FIG. 59, graph (f)).

### Experimental Example 19

### Identification of Possibility as a Nano-Contrast Agent Via an Animal Model

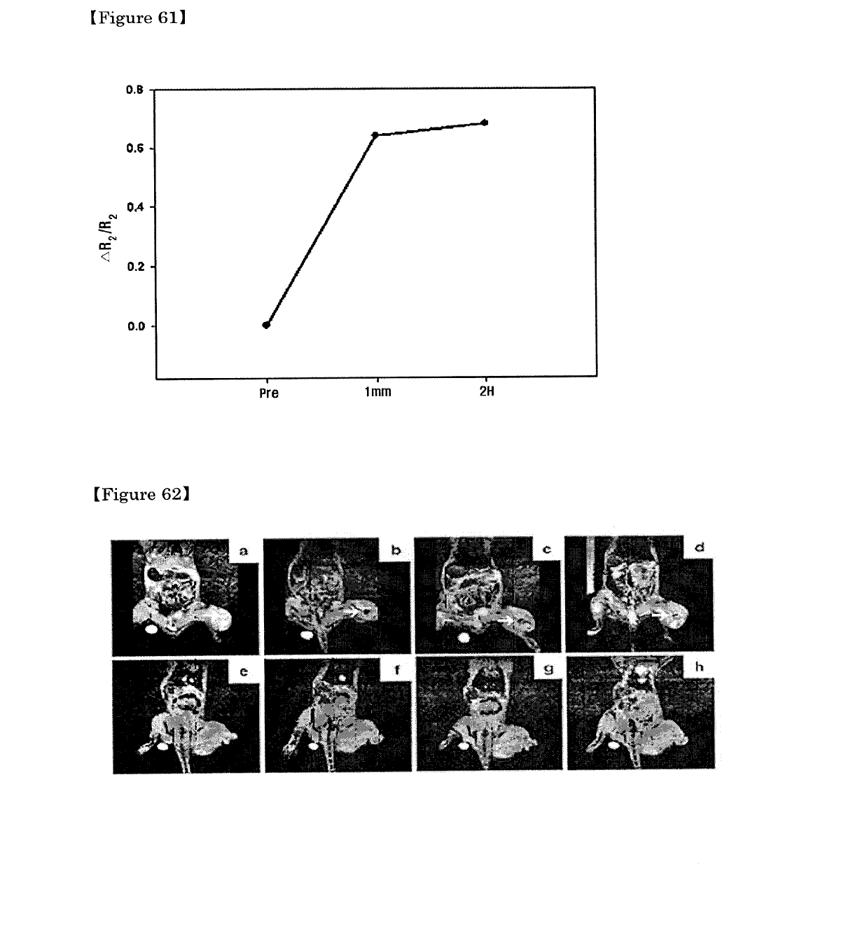

In vivo experiment was subjected, using a nude mouse as an animal model. NIH3T6.7 cells positive against antibody were into the mouse to express cancer cells. After 2 days, the contrast agent prepared in Example 6 was injected therein, when the size of cancer cells was 10 mm. MRIs before and after injection were depicted in FIG. 60. In FIG. 60, there are MRIs before injection FIG. 60, picture (a), just after injection FIG. 60, picture (b), and two hours after injection FIG. 60, picture (c), of the contrast agent. As represented in FIG. 60, it could be identified that images of liver and cancer cells were apparently changed. In addition, the change from before injection to 2 hours over time was depicted in FIG. 61. As depicted in FIG. 61, it could be identified that the T2 values after injection were highly changed.

### Experimental Example 20

### Identification of Possibility in the Magnetic Nanocomposite as a Nano-Contrast Agent Via an Animal Model

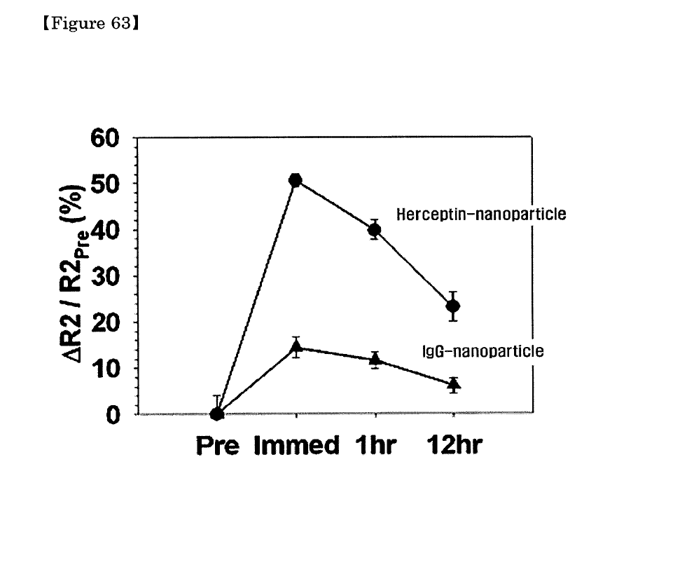

To know whether the magnetic nanocomposite may trace cancer cells, NIH3T6.7 cells were implanted into thighs of a group of nude mice. 1.5 T system (Intera; Philips Medical Systems, Best, The Netherlands) was used for the contrasting effect of MRI, employing micro-47 coil. Coronal images were obtained with Fast Field Echo (FFE) pulse sequence. Specific parameters were as follows: resolution 156 156 μm, slice thickness 0.6 mm, TE=20 ms, TR=400 ms, number of image excitation 1, time of image acquisition 6 minutes. The contrasting effect was identified over time. Images were obtained, at preliminary (Pre) injection, immediately (Immed) after injection into a tail vein, 4 hours after injection, and 12 hours after injection, of the nanocomposite prepared in Example 10, B., and the results were depicted in pictures (a)-(h) of FIG. 62 and FIG. 63. It could be identified that the very high contrasting effect was represented in the thigh of nude mouse 12 hours after injection, and that the contrasting effect of IgG-magnetic nanocomposite without herceptin was lowered. As a result, it could be identified that herceptin-magnetic nanocomposite was selectively targeted at cancer cells.

### INDUSTRIAL APPLICABILITY

The magnetic nanocomposite according to the present invention, covered with the amphiphilic compound having hydrophobic domains and a hydrophilic domains may be used in a contrast agent for high sensitive MRI, an intelligent contrast agent for diagnosing cancer by binding to the binding parts materials that may specifically be bound to tumor markers, a drug delivery system for diagnosis and treatment of cancer by polymerizing or enclosing a drug in the hydrophobic domains, and a formulation for separating cells and proteins using magnetism by binding an antibody or a protein specific to surface antigens of functional cells, stem cells or cancer cells thereto.

We claim:

1. A method of preparing a magnetic nanocomposite comprising the steps of:
A) reacting precursors of nanoparticles with an organic surface stabilizer in presence of a solvents and thermolyzing a resulting reactant to synthesize magnetic nanoparticles;
B) adding an amphiphilic compound having a hydrophobic domain and a hydrophilic domain to the magnetic nanoparticles to form a magnetic nanocomposite, the magnetic nanocomposite comprising a core containing one or more magnetic nanoparticles distributed in the hydrophobic domain of the amphiphilic compound and a shell containing the hydrophilic domain of the amphiphilic compound, wherein the hydrophobic domain is bound to a surface of the magnetic nanoparticles by physical bond;
C) binding a tissue-specific binding substance to one or more binding parts for a hydrophilic active ingredient in the hydrophilic domain; and
D) binding or enclosing a pharmaceutically active ingredient in the hydrophobic domain.

2. The method for preparing a magnetic nanocomposite according to claim 1,
wherein the step B) comprises the steps of:
a) dissolving nanoparticles in an organic solvent to prepare an oil phase;
b) dissolving an amphiphilic compound in an aqueous solvent to prepare an aqueous phase;
c) mixing the oil phase and the aqueous phase to form an emulsion; and
d) separating the oil phase from the emulsion.

3. The method for preparing a magnetic nanocomposite according to claim 2, wherein the solvent is selected from the group consisting of an ether compound, a heterocyclic compound, an aromatic compound, a sulfoxide compound, an amide compound, an alcohol, a hydrocarbon and water.

4. The method for preparing a magnetic nanocomposite according to claim 3,
wherein the solvent is octyl ether, butyl ether, hexyl ether, decyl ether, pyridine, tetrahydrofuran(THF), dimethylformamide(DMF), octyl alcohol, decanol, pentane, hexane, heptanes, octane, decane, dodecane, tetradecane, hexadecane, or water.

5. The method for preparing the magnetic nanocomposite according to claim 1, wherein the step C) comprises the steps of:
a) introducing one or more binding parts for a hydrophilic active ingredient into some of the hydrophilic domain, using a cross linking agent; and
b) binding the one or more binding parts for the hydrophilic active ingredient and a tissue specific binding substance.

6. The method for preparing the magnetic nanocomposite according to claim 1, wherein the step D) comprises one of the following steps:
a) introducing one or more binding parts for a hydrophobic active ingredient into some of the hydrophobic domain, using a cross linking agent; and binding the one or more binding parts for the hydrophobic active ingredient and the pharmaceutically active ingredient, and
b) enclosing the pharmaceutically active ingredient in the hydrophobic domain by dissolving the pharmaceutically active ingredient together with nanoparticles in step B).

7. The method for preparing the magnetic nanocomposite according to claim 1, wherein the magnetic nanoparticle is a magnetic material selected from the group consisting of Co, Mn, Fe, Ni, Gd, Mo, MM′₂O₄, and MₓO_{y} (where each M and M′ independently represents Co, Fe, Ni, Mn, Zn, Gd, or Cr, 0<x≦3, and 0<y≦5).

8. The method for preparing the magnetic nanocomposite according to claim 1, wherein the magnetic nanoparticle is bound to one or more organic surface stabilizers selected from the group consisting of alkyl trimethylammonium halide, a saturated or unsaturated fatty acid, trialkylphosphine, trialkylphosphine oxide, alkyl amine, alkyl thiol, sodium alkyl sulfate, and sodium alkyl phosphate.

9. The method for preparing the magnetic nanocomposite according to claim 1, wherein the hydrophobic domain is a saturated or unsaturated fatty acid, or a hydrophobic polymer.

10. The method for preparing the magnetic nanocomposite according to claim 1, wherein the hydrophilic domain is a biodegradable polymer.

11. The method for preparing the magnetic nanocomposite according to claim 10, wherein the biodegradable polymer is selected from the group consisting of polyalkyleneglycol (PAG), polyetherimide (PEI), polyvinylpyrrolidone (PVP), a hydrophilic polyamino acid and a hydrophilic vinyl based polymer or combinations thereof.

12. The method for preparing the magnetic nanocomposite according to claim 1, wherein the tissue-specific binding substance is selected from the group consisting of an antigen, an antibody, RNA, DNA, hapten, avidin, streptavidin, neutravidin, protein A, protein G, lectin, selectin, a radioisotope labeled component, and a material that is capable of specifically binding to a tumor marker or combinations thereof.

13. The method for preparing the magnetic nanocomposite according to claim 1, wherein the amphiphilic compound is monomethoxypolyethyleneglycol-polylactide-co-glycolide copolymer, or monomethoxypolyethyleneglycol-lauric acid copolymer.

14. The method for preparing the magnetic nanocomposite according to claim 1, wherein the pharmaceutically active ingredient is selected from the group consisting of an anticancer agent, an antibiotic, a hormone, a hormone antagonist, interleukin, interferon, a growth factor, a tumor necrosis factor, endotoxin, lymphotoxin, eurokinase, streptokinase, a tissue plasminogen activator, a protease inhibitor, alkylphosphocholine, a radioisotope labeled component, a surfactant, a cardiovascular system drug, a gastrointestinal system drug and a nervous system drug or combinations thereof.

15. A magnetic nanocomposite prepared by the method according to claim 1.
